(19) United States
(12) Patent Application Publication
(10) Pub. No.: US 2026/0182928 A1
(43) Pub. Date: Jul. 2, 2026

(54) METHOD AND DEVICE FOR PERFORMING NEUROMUSCULAR SIGNAL ANALYSIS

(71) Applicant: EXOSYSTEMS INC., Seongnam-si Gyeonggi-do (KR)

(72) Inventors: Hooman LEE, Seongnam-si Gyeonggi-do (KR); Sangui CHOI, Seoul (KR); Kwangsub SONG, Gwangju-si Gyeonggi-do (KR)

(73) Assignee: EXOSYSTEMS INC., Seongnam-si Gyeonggi-do (KR)

(21) Appl. No.: 19/550,450

(22) Filed: Feb. 26, 2026

Related U.S. Application Data
(63) Continuation of application No. PCT/KR2024/016045, filed on Oct. 22, 2024.

(30) Foreign Application Priority Data
Oct. 27, 2023 (KR) 10-2023-0145553
Nov. 10, 2023 (KR) 10-2023-0155237
Mar. 27, 2024 (KR) 10-2024-0041921
May 20, 2024 (KR) 10-2024-0064947
May 31, 2024 (KR) 10-2024-0071359
May 31, 2024 (KR) 10-2024-0071394

(51) Int. Cl. A61B 5/00; A61B 5/11; G16H 10/60

(52) U.S. Cl. CPC A61B 5/7267; A61B 5/1104; A61B 5/7257; G16H 10/60

(57) ABSTRACT

The present disclosure relates to a method of evaluating a neuromuscular system, the method comprising: obtaining a neuromuscular system signal; dividing the neuromuscular system signal into a plurality of segment units based on frequency; extracting a feature vector for each of the divided plurality of segments; and performing neuromuscular system condition evaluation by inputting the extracted feature vector into an artificial intelligence model trained through artificial intelligence-based model training.

### CROSS-REFERENCE TO RELATED APPLICATIONS

This application is a continuation of International Application No. PCT/KR2024/016045 filed on Oct. 22, 2024, which claims priority to Korean Patent Application No. 10-2023-0145553 filed on Oct. 27, 2023, Korean Patent Application No. 10-2023-0155237 filed on Nov. 10, 2023, Korean Patent Application No. 10-2024-0041921 filed on Mar. 27, 2024, Korean Patent Application No. 10-2024-0064947 filed May 20, 2024, Korean Patent Application No. 10-2024-0071359 filed on May 31, 2024, and Korean Patent Application No. 10-2024-0071394 filed on May 31, 2024, the entire contents of which are herein incorporated by reference.

### TECHNICAL FIELD

The present disclosure relates to a method and device for performing neuromuscular system signal analysis. More specifically, the present disclosure relates to a method and device for performing neuromuscular system condition evaluation by analyzing response signals based on electrical stimulation. The present disclosure also relates to a method and device for analyzing neuromuscular system signals for ECF (electrical current frequency) stimulation.

### BACKGROUND ART

Sarcopenia refers to a condition characterized by a reduction in muscle mass, muscle strength, and muscle function. Although the causes of sarcopenia vary among individuals, the most common causes include reduced protein intake, insufficient physical activity, and poor exercise habits. In particular, sarcopenia frequently results from inadequate intake and absorption of essential amino acids. Another common cause of sarcopenia is hormone deficiency associated with aging. In addition to diseases originating in the neuromuscular system itself, sarcopenia frequently occurs as a secondary condition due to acute and chronic diseases such as diabetes, infectious diseases, and cancer, as well as degenerative diseases such as spinal stenosis. It is known that sarcopenia occurs at a high frequency in cases of chronic diseases affecting the heart, lungs, and kidneys, as well as hormonal disorders. Symptoms of sarcopenia include decreased muscle strength, lower limb weakness, and fatigue. In many cases, decreased muscle strength precedes the onset of sarcopenia. When decreased muscle strength or sarcopenia is present, the most important step is to identify factors contributing to symptom aggravation, confirm any comorbidities, and eliminate the underlying causes. Patients with sarcopenia experience a slower gait and reduced muscle endurance, making daily activities difficult and frequently requiring assistance from others. In addition, they are prone to osteoporosis, falls, and fractures. As the buffering capacity of muscles for blood and hormones diminishes, basal metabolic rate decreases, chronic disease management becomes more difficult, and diabetes and cardiovascular disease may be readily aggravated. In order to diagnose conditions such as sarcopenia, it is important to accurately assess the condition of muscles. However, at present, neuromuscular system condition evaluation is primarily performed by specialists using expensive equipment. Accordingly, there is a need for a technology capable of accurately performing neuromuscular system condition evaluation even at home or by non-specialists.

### PRIOR ART DOCUMENTS

### Patent Documents

(Patent Document 1) Korean Patent Application Publication No. 10-2018-0074597

### Technical Problem

The present disclosure is directed to solving the above-described technical problems. The present disclosure is capable of performing neuromuscular system condition evaluation by analyzing response signals based on electrical stimulation, and is capable of analyzing neuromuscular system signals for ECF (electrical current frequency) stimulation.

### Technical Solution

In order to solve the above-described problems, one embodiment of the present disclosure provides a method, performed by a device, of evaluating a neuromuscular system, the method comprising: obtaining a neuromuscular system signal; performing a Fourier Transform on the neuromuscular system signal to generate FT neuromuscular system signal information; applying a logarithmic function to the FT neuromuscular system signal information to calculate a log spectrum of the neuromuscular system signal; performing an Inverse Fourier Transform on the calculated log spectrum to obtain CA (cepstrum analysis) coefficient information; obtaining a feature vector based on the CA coefficient information; and evaluating the neuromuscular system based on the obtained feature vector.

In addition, the neuromuscular system signal may be a signal generated in response to an electrical stimulation.

In addition, obtaining the feature vector may comprise generating preprocessed CA coefficient information by removing, from the CA coefficient information, a FF (fundamental frequency) component corresponding to a frequency of the electrical stimulation, and obtaining a feature vector based on the preprocessed CA coefficient information.

In addition, obtaining the feature vector may further comprise generating preprocessed CA coefficient information by further removing, from the CA coefficient information, an HF (harmonic frequency) component generated by the electrical stimulation, and obtaining a feature vector based on the preprocessed CA coefficient information.

In addition, obtaining the CA (cepstrum analysis) coefficient information by performing an Inverse Fourier Transform may comprise obtaining the CA coefficient information based on a complex value among a complex value and a real value.

In addition, obtaining the CA (cepstrum analysis) coefficient information by performing an Inverse Fourier Transform may comprise obtaining the CA coefficient information based on a real value among a complex value and a real value.

In addition, obtaining the CA (cepstrum analysis) coefficient information by performing an Inverse Fourier Transform may comprise obtaining the CA coefficient information based on a value obtained by squaring each of a complex value and a real value, adding the squared values, and applying a square root operation.

In addition, evaluating the neuromuscular system based on the obtained feature vector may comprise evaluating the neuromuscular system by inputting the obtained feature vector into an artificial intelligence model.

In addition, the artificial intelligence model may be an artificial intelligence model that evaluates at least one of sarcopenia diagnosis, muscle strength evaluation, muscle endurance evaluation, gait speed evaluation, and muscle mass evaluation.

In addition, obtaining the feature vector may comprise obtaining temporal change statistics information of the preprocessed CA coefficient information, and obtaining a feature vector based on the temporal change statistics information.

In addition, the temporal change statistics information may be determined based on at least one of a maximum value, a minimum value, a mean value, a median value, a standard deviation value, a variance value, and an IQR (inter-quartile range) value of the preprocessed CA coefficient information over time.

In order to solve the above-described problems, another embodiment of the present disclosure provides a device for evaluating a neuromuscular system, the device comprising: a muscle signal acquisition unit configured to acquire a neuromuscular system signal; a cepstrum analysis unit configured to perform a Fourier Transform on the neuromuscular system signal to generate FT neuromuscular system signal information, calculate a log spectrum of the neuromuscular system signal by applying a logarithmic value to the FT neuromuscular system signal information, and perform an Inverse Fourier Transform on the calculated log spectrum to obtain CA (cepstrum analysis) coefficient information; a feature vector acquisition unit configured to obtain a feature vector based on the CA coefficient information; and a muscle condition evaluation unit configured to evaluate the neuromuscular system based on the obtained feature vector.

In addition, the neuromuscular system signal may be a signal generated in response to an electrical stimulation.

In addition, the feature vector acquisition unit may be configured to generate preprocessed CA coefficient information by removing, from the CA coefficient information, a FF (fundamental frequency) component corresponding to a frequency of the electrical stimulation, and obtain a feature vector based on the preprocessed CA coefficient information.

In addition, the feature vector acquisition unit may be further configured to generate preprocessed CA coefficient information by further removing, from the CA coefficient information, an HF (harmonic frequency) component generated by the electrical stimulation, and obtain a feature vector based on the preprocessed CA coefficient information.

In order to solve the above-described problems, another embodiment of the present disclosure provides a computer program stored on a computer-readable storage medium, wherein the computer program comprises instructions for causing one or more processors to perform neuromuscular system evaluation, the instructions comprising: obtaining a neuromuscular system signal; performing a Fourier Transform on the neuromuscular system signal to generate FT neuromuscular system signal information; applying a log value to the FT neuromuscular system signal information to calculate a log spectrum of the neuromuscular system signal; performing an Inverse Fourier Transform on the calculated log spectrum to obtain CA (cepstrum analysis) coefficient information; obtaining a feature vector based on the CA coefficient information; and evaluating the neuromuscular system based on the obtained feature vector.

In addition, the neuromuscular system signal may be a signal generated in response to an electrical stimulation.

In addition, obtaining the feature vector may comprise generating preprocessed CA coefficient information by removing, from the CA coefficient information, a FF (fundamental frequency) component corresponding to a frequency of the electrical stimulation, and obtaining a feature vector based on the preprocessed CA coefficient information.

In addition, obtaining the feature vector may further comprise generating preprocessed CA coefficient information by further removing, from the CA coefficient information, an HF (harmonic frequency) component generated by the electrical stimulation, and obtaining a feature vector based on the preprocessed CA coefficient information.

In addition, obtaining the CA (cepstrum analysis) coefficient information by performing an Inverse Fourier Transform may comprise obtaining the CA coefficient information based on a complex value among a complex value and a real value.

### Advantageous Effects

The method and device according to the embodiments of the present disclosure are capable of performing neuromuscular system condition evaluation by analyzing response signals based on electrical stimulation, and are capable of analyzing neuromuscular system signals for ECF (electrical current frequency) stimulation.

### BRIEF DESCRIPTION OF THE DRAWINGS

FIG. 1A and FIG. 1B are diagrams for describing a method of applying electrical stimulation to a user's body and obtaining a neuromuscular system signal.

FIG. 2 is a diagram for describing a device (100) according to an embodiment of the present disclosure.

FIG. 3 is a diagram for describing a method of evaluating a neuromuscular system condition according to an embodiment of the present disclosure.

FIG. 4 is a diagram for describing functions of a processor according to an embodiment of the present disclosure.

FIG. 5 is a diagram for describing a method of dividing an acquired neuromuscular system signal into a plurality of segments according to an embodiment of the present disclosure.

FIG. 6 is a diagram for describing a method of generating an envelope for each of a plurality of segments according to an embodiment of the present disclosure.

FIG. 7 is a diagram for describing a method of applying interpolation to generate an envelope according to an embodiment of the present disclosure.

FIG. 8 is a diagram for describing a method of extracting a feature vector from an envelope according to an embodiment of the present disclosure.

FIG. 9 is a diagram for describing a method of generating an artificial intelligence model according to an embodiment of the present disclosure.

FIG. 10 is a schematic diagram illustrating an artificial neural network according to an embodiment of the present disclosure.

FIG. 11 is a general schematic diagram of an exemplary device (100) in which embodiments of the present disclosure may be implemented.

FIG. 12 is a diagram for describing a method of obtaining a neuromuscular system signal and removing noise according to an embodiment of the present disclosure.

FIG. 13 is a diagram for describing functions of a processor according to an embodiment of the present disclosure.

FIG. 14 is a diagram for describing in detail a process of removing noise from a neuromuscular system signal.

FIG. 15 is a diagram for describing a method of evaluating a neuromuscular system according to another embodiment of the present disclosure.

FIG. 16 is a diagram for describing functions of a processor according to an embodiment of the present disclosure.

FIG. 17, FIG. 18, and FIG. 19 are reference diagrams for describing signal information analysis according to an embodiment of the present disclosure.

FIG. 20 is a diagram for describing an experimental case according to an embodiment of the present disclosure.

FIG. 21 is a diagram for describing a method of evaluating a neuromuscular system according to an embodiment of the present disclosure.

FIG. 22 is a diagram for describing a method by which the device (100) generates comparison data using a signal acquired by a first stimulation and a signal acquired by a last stimulation among one set of electrical stimulations.

FIGS. 23A and 23B are diagrams illustrating a trend of a neuromuscular system signal according to an embodiment of the present disclosure.

FIG. 24 is a diagram for describing zero-crossing data according to an embodiment of the present disclosure.

FIG. 25 is a diagram for describing slope sign change data according to an embodiment of the present disclosure.

FIG. 26 is a diagram for exemplarily describing a method of measuring variance data.

FIG. 27 is a diagram for describing an embodiment in which a device applies a plurality of sets of electrical stimulations according to an embodiment of the present disclosure.

FIG. 28 is a diagram for comparing a neuromuscular system signal produced by a first stimulation with a neuromuscular system signal produced by a last stimulation according to an embodiment of the present disclosure.

FIG. 29 is a diagram for describing a method of evaluating a neuromuscular system according to an embodiment of the present disclosure.

FIG. 30 is a diagram for describing an artificial intelligence model according to an embodiment of the present disclosure.

FIG. 31 is a diagram for describing a pattern extraction method according to an embodiment of the present disclosure.

FIG. 32 is a diagram for describing a method of obtaining a positive peak and a negative peak.

FIG. 33 is a diagram for describing a method of obtaining an envelope.

FIG. 34 is a diagram for describing a method of extracting feature information based on a generated envelope.

FIG. 35 is a diagram for describing a method of evaluating a neuromuscular system according to an embodiment of the present disclosure.

FIG. 36 is a diagram for describing acquisition of a neuromuscular system signal using muscle electrical stimulation and evaluation electrical stimulation.

FIG. 37 is a diagram for describing a method of obtaining intermediate evaluation data according to an embodiment of the present disclosure.

FIG. 38 is a diagram for describing a method of applying a cumulative function to intermediate evaluation data.

FIG. 39A is a diagram illustrating intermediate evaluation data according to an embodiment of the present disclosure.

FIG. 39B and FIG. 39C are diagrams illustrating an embodiment in which a cumulative function is applied to intermediate evaluation data according to an embodiment of the present disclosure.

FIG. 40 is a diagram for describing normalization performed on data to which a cumulative function has been applied according to an embodiment of the present disclosure.

FIG. 41 is a diagram for describing a method of applying evaluation electrical stimulation and obtaining a corresponding neuromuscular system signal according to an embodiment of the present disclosure.

### DETAILED DESCRIPTION OF THE INVENTION

Various embodiments will now be described with reference to the drawings. In the present specification, various descriptions are presented in order to provide an understanding of the present disclosure. However, it will be apparent that these embodiments may be practiced without these specific descriptions.

As used herein, the terms “component,” “module,” “system,” and the like refer to a computer-related entity, hardware, firmware, software, a combination of software and hardware, or an execution of software. For example, a component may be, but is not limited to, a process running on a processor, a processor, an object, an execution thread, a program, and/or a computer. For example, both an application running on a computing device and the computing device itself may be a component. One or more components may reside within a processor and/or an execution thread. A component may be localized within one computer. A component may be distributed between two or more computers. In addition, such components may execute from various computer-readable media having various data structures stored therein. Components may communicate via local and/or remote processes in accordance with, for example, a signal having one or more data packets (e.g., data from one component interacting with another component in a local system or a distributed system, and/or data transmitted over a network such as the Internet with another system).

In addition, the term “or” is intended to mean an inclusive “or” rather than an exclusive “or.” That is, unless otherwise specified or clear from context, “X uses A or B” is intended to mean one of the natural inclusive substitutions. That is, if X uses A; X uses B; or X uses both A and B, “X uses A or B” may apply to any of these cases. Furthermore, the term “and/or” as used herein should be understood to refer to and include all possible combinations of one or more of the listed related items.

In addition, the terms “comprises” and/or “comprising” should be understood to mean that the corresponding feature and/or component is present. However, the terms “comprises” and/or “comprising” should be understood as not excluding the presence or addition of one or more other features, components, and/or groups thereof. Furthermore, unless otherwise specified or clear from context as indicating a singular form, the singular in this specification and claims should generally be construed to mean “one or more.”

In addition, the term “at least one of A or B” should be construed to mean “a case including only A,” “a case including only B,” or “a case in which A and B are combined.”

Those skilled in the art should further recognize that the various illustrative logical blocks, configurations, modules, circuits, means, logics, and algorithm steps described in connection with the embodiments disclosed herein may be implemented in electronic hardware, computer software, or combinations of both. To clearly illustrate this interchangeability of hardware and software, various illustrative components, blocks, configurations, means, logics, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application. However, such implementation decisions should not be interpreted as causing a departure from the scope of the present disclosure.

As used herein, feature information refers to data representing inherent attributes or characteristics of a target object or phenomenon, and means information extracted or generated from input data in artificial intelligence and machine learning systems. Feature information may exist in various forms, including numerical, categorical, text-based, and vector forms. Furthermore, as used herein, the term “feature vector” is not limited to a vector form and is used with the meaning of feature information.

The description of the presented embodiments is provided to enable any person skilled in the art of the present disclosure to make or use the present disclosure. Various modifications to these embodiments will be apparent to those skilled in the art of the present disclosure. The general principles defined herein may be applied to other embodiments without departing from the scope of the present disclosure. Therefore, the present disclosure is not limited to the embodiments presented herein. The present disclosure should be interpreted in the broadest scope consistent with the principles and novel features presented herein.

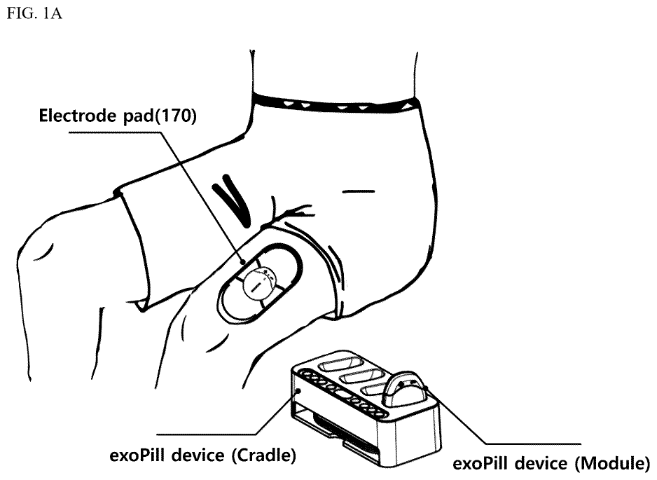

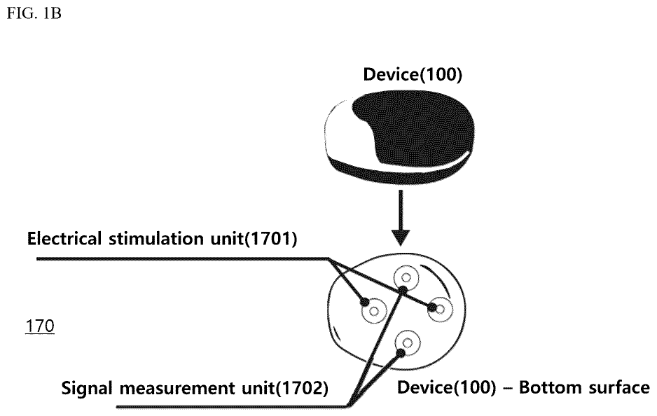

FIG. 1A and FIG. 1B are diagrams for describing a method of applying electrical stimulation to a user's body and obtaining a neuromuscular system signal.

Referring to FIG. 1A and FIG. 1B, the device (100) may comprise an electrical stimulation and measurement unit (170). The electrical stimulation and measurement unit (170) may be connected to other components of the device (100) via a wired or wireless connection. The electrical stimulation and measurement unit (170) may apply electrical stimulation (hereinafter, “ES”) to body muscles such as leg muscles, back muscles, and chest muscles, measure an electrical stimulation-based neuromuscular system signal, and provide the measured values to the device (100). Here, the electrical stimulation-based neuromuscular system signal may comprise electromyography (hereinafter, “EMG”) data obtained by applying electrical stimulation to muscles. The EMG data may comprise electromyography (EMG) data measured by an EMG sensor. In this case, the acquired neuromuscular system signal may be referred to as a stimulated muscle contraction signal (hereinafter, “SMCS”).

According to an embodiment of the present disclosure, the electrical stimulation applied to muscles may be provided as multi-frequency electrical stimulation. As a result, the EMG data may comprise a multi-frequency stimulated muscle contraction signal (SMCS).

The device (100) may receive a neuromuscular system signal from the electrical stimulation and measurement unit (170) and analyze the received neuromuscular system signal. The device (100) may remove noise electrical signals contained in the stimulated muscle contraction signal (SMCS). A reference signal for training and performance evaluation of the artificial intelligence model may be measured using torque equipment for measuring muscle strength and muscle endurance.

In addition, the device (100) may extract a feature vector representing characteristics of muscle strength and muscle endurance from the stimulated muscle contraction signal (SMCS). The device (100) may perform artificial intelligence (AI) model training using deep learning or a support vector machine (SVM). The device (100) may generate a deep learning model and process the feature vector using the deep learning model. The device (100) may classify the degree of muscle strength and muscle endurance based on the feature vector. In addition, the device (100) may diagnose sarcopenia based on the feature vector.

According to an embodiment of the present disclosure, the electrical stimulation and measurement unit (170) may be implemented in various forms, such as a belt form, a pad form, or an electrode form. In addition, according to another embodiment, the electrical stimulation and measurement unit (170) may be implemented as a small body-attachable device.

FIG. 1A illustrates an electrical stimulation and measurement unit (170) in a pad form, and FIG. 1B illustrates an electrical stimulation and measurement unit (170) incorporated in the device (100). The electrical stimulation and measurement unit (170) may be worn (or attached) to a user's body (e.g., thigh). The electrical stimulation and measurement unit (170) may apply electrical stimulation (ES) to a user's body muscles (e.g., thigh muscles) and measure a response signal.

Referring to FIG. 1B, the electrical stimulation and measurement unit (170) may comprise an electrical stimulation unit (1701) and a signal measurement unit (1702). The electrical stimulation unit (1701) may be connected to a stimulation signal generation circuit (not shown). The stimulation signal generation circuit may be provided in the device (100). The electrical stimulation unit (1701) may apply electrical stimulation (ES) to the thigh. The electrical stimulation unit (1701) may apply electrical stimulation (ES) to a user's muscles in order to collect biosignals (e.g., EMG signals) of the user.

The stimulation signal generation circuit may generate a signal for electrical stimulation (ES). The stimulation signal generation circuit may comprise an ES generator for applying electrical stimulation to thigh muscles. The electrical stimulation unit (1701) may apply the electrical stimulation signal generated by the ES generator to the thigh muscles using a thigh electrical stimulation pad. The intensity, frequency, current, or waveform of the electrical stimulation signal may be adjusted according to the degree of muscle stimulation of the user. Here, the electrical stimulation applied to muscles may be provided as multi-frequency electrical stimulation. The signal measurement unit (1702) may be connected to (or include) a neuromuscular system measurement sensing circuit (not shown). The neuromuscular system measurement sensing circuit may be an electromyography (EMG) measurement sensing circuit. The neuromuscular system measurement sensing circuit may comprise an EMG sensor for thigh EMG measurement sensing. When the electrical stimulation applied to muscles is multi-frequency electrical stimulation, the EMG data measured by the EMG sensor may be provided as a multi-frequency stimulated muscle contraction signal (SMCS). Meanwhile, the signal measurement unit (1702) may provide measurement information (i.e., ES-based IR (electrical stimulation-based impedance response)) to the device (100).

When referring to FIG. 1B, the electrical stimulation and measurement unit (170) may be implemented in an electrode form. When referring to FIG. 1A, the electrical stimulation unit (1701) may comprise an electrical stimulation pad. The electrical stimulation pad may be used in a wet form, either as a disposable or reusable pad. Alternatively, the electrical stimulation pad may be manufactured using a dry high-adhesion material to transmit biosignals of the user or electrical stimulation signals of innervated muscles. For example, the electrical stimulation pad may be manufactured as a conductive dry adhesive electrode pad using carbon nano-materials.

The signal measurement unit (1702) may acquire measurement information (e.g., a multi-frequency stimulated muscle contraction signal (SMCS)) through the electrical stimulation measurement pad. The neuromuscular system measurement sensing circuit may be an EMG measurement sensing circuit. The neuromuscular system measurement sensing circuit may comprise an EMG sensor for thigh EMG measurement sensing.

When referring to FIG. 1A, the applicant's Exofil device (module) may be an example of the device (100). The Exofil device (module) may be received in and charged by the Exofil device (cradle).

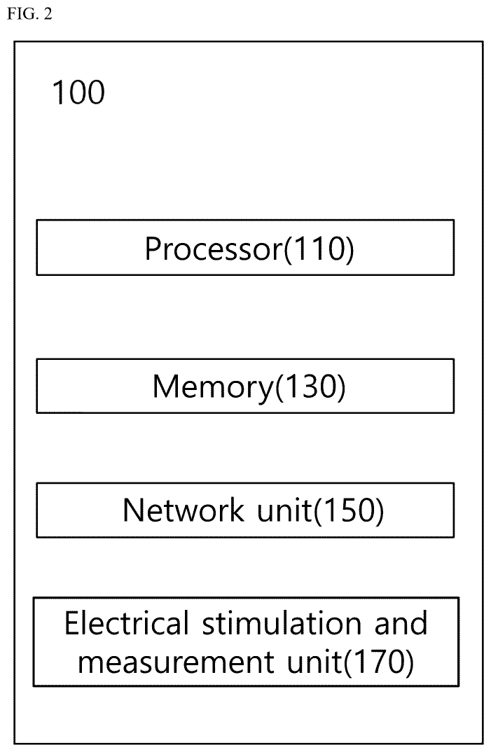

FIG. 2 is a diagram for describing a device (100) according to an embodiment of the present disclosure.

FIG. 2 shows a simplified example of the configuration of the device (100). In an embodiment of the present disclosure, the device (100) may comprise other components for performing the computing environment of the device (100), and only some of the disclosed components may constitute the device (100).

In addition, the device (100) may be implemented in various forms. For example, the device (100) may be implemented as a server or as a small electrical device, without being limited thereto.

The device (100) may comprise a processor (110), a memory (130), a network unit (150), and an electrical stimulation and measurement unit (170). In this case, each of the components may be included and implemented in the device (100) as a single piece of hardware. In addition, each of the components may be implemented as separate hardware, and the separate hardware components may communicate via wired or wireless communication.

The processor (110) may be composed of one or more cores, and may comprise processors for data analysis and deep learning, such as a central processing unit (CPU), a general purpose graphics processing unit (GPGPU), and a tensor processing unit (TPU) of a computing device. The processor (110) may read a computer program stored in the memory (130) and perform data processing for machine learning according to an embodiment of the present disclosure. According to an embodiment of the present disclosure, the processor (110) may perform operations for training a neural network. The processor (110) may perform computations for neural network training, such as processing of input data for deep learning (DL), feature extraction from input data, error calculation, and weight update of the neural network using backpropagation. At least one of the CPU, GPGPU, and TPU of the processor (110) may process the training of a network function. For example, a CPU and a GPGPU may together process the training of a network function and data classification using the network function. In addition, in an embodiment of the present disclosure, processors of a plurality of computing devices may be used together to process the training of a network function and data classification using the network function. In addition, a computer program executed in a computing device according to an embodiment of the present disclosure may be a CPU-, GPGPU-, or TPU-executable program.

According to an embodiment of the present disclosure, the memory (130) may store any form of information generated or determined by the processor (110) and any form of information received by the network unit (150).

According to an embodiment of the present disclosure, the memory (130) may comprise at least one type of storage medium among a flash memory type, a hard disk type, a multimedia card micro type, a card-type memory (e.g., SD or XD memory), random access memory (RAM), static random access memory (SRAM), read-only memory (ROM), electrically erasable programmable read-only memory (EEPROM), programmable read-only memory (PROM), magnetic memory, a magnetic disk, and an optical disk. The device (100) may also operate in association with web storage that performs the storage function of the memory (130) over the Internet. The foregoing description of the memory is illustrative only, and the present disclosure is not limited thereto.

The network unit (150) according to an embodiment of the present disclosure may use various wired communication systems such as a public switched telephone network (PSTN), x digital subscriber line (xDSL), rate adaptive DSL (RADSL), multi rate DSL (MDSL), very high speed DSL (VDSL), universal asymmetric DSL (UADSL), high bit rate DSL (HDSL), and a local area network (LAN).

In addition, the network unit (150) presented herein may use various wireless communication systems such as code division multiple access (CDMA), time division multiple access (TDMA), frequency division multiple access (FDMA), orthogonal frequency division multiple access (OFDMA), single carrier-FDMA (SC-FDMA), and other systems.

In the present disclosure, the network unit (150) may be configured regardless of its communication mode, such as wired or wireless, and may be configured with various communication networks such as a personal area network (PAN) and a wide area network (WAN). In addition, the network may be the well-known World Wide Web (WWW), and may use wireless transmission technologies used for short-range communication, such as infrared data association (IrDA) or Bluetooth.

The electrical stimulation and measurement unit (170) according to an embodiment of the present disclosure has been described above with reference to FIG. 1A and FIG. 1B.

According to an embodiment for solving one of the technical problems of the present disclosure, a method and device may be provided for evaluating a neuromuscular system condition by analyzing a response signal based on electrical stimulation using an artificial intelligence learning model.

Specifically, one embodiment of the present disclosure for solving the above-described problems may provide a method, performed by a device, of performing neuromuscular system condition evaluation, the method comprising: obtaining a neuromuscular system signal; dividing the neuromuscular system signal into a plurality of segments based on frequency; extracting a feature vector for each of the divided plurality of segments; and performing neuromuscular system condition evaluation by inputting the extracted feature vector into an artificial intelligence model trained through artificial intelligence-based model training. In addition, the extracting of the feature vector may comprise: converting the neuromuscular system signal into a time-frequency domain for each of the divided plurality of segments; generating an envelope from the converted neuromuscular system signal for each of the divided plurality of segments; and analyzing a pattern of the generated envelope to obtain a feature vector on a segment basis. In addition, the generating of the envelope may comprise: obtaining an envelope peak using a positive peak and a negative peak; and generating an envelope by interpolating between the envelope peaks. In addition, the obtaining of the envelope peak may obtain an envelope peak by adding magnitude values of adjacent positive and negative peaks. In addition, the obtaining of the envelope peak may obtain an envelope peak by calculating an average of magnitude values of adjacent positive and negative peaks. In addition, the generating of the envelope may comprise: obtaining a positive peak as an envelope peak; and generating an envelope by interpolating between the envelope peaks. In addition, the generating of the envelope may comprise: obtaining a negative peak as an envelope peak; and generating an envelope by interpolating between the envelope peaks. In addition, the analysis of the pattern of the envelope may be performed by at least one of sample entropy analysis, permutation entropy analysis, standard deviation signal analysis, square root analysis, auto-correlation analysis, cardinality analysis, and Poincaré plot analysis. In addition, the evaluating of the muscle may diagnose sarcopenia by inputting the feature vector into an artificial intelligence model for diagnosing sarcopenia. In addition, the evaluating of the muscle may evaluate muscle strength by inputting the feature vector into an artificial intelligence model for evaluating muscle strength. In addition, the evaluating of the muscle may evaluate muscle endurance by inputting the feature vector into an artificial intelligence model for evaluating muscle endurance. In addition, the neuromuscular system signal in response to the stimulation may be a response signal from the body when multi-frequency stimulation is applied to the body.

In addition, another embodiment for solving the above-described technical problems may provide a device for performing neuromuscular system condition evaluation, the device comprising: a neuromuscular system signal acquisition unit configured to acquire a neuromuscular system signal in response to a stimulation; a segment division unit configured to divide the neuromuscular system signal into a plurality of segments based on frequency; a feature vector extraction unit configured to extract a feature vector for each of the divided plurality of segments; and a neuromuscular system condition evaluation unit configured to perform neuromuscular system condition evaluation by inputting the extracted feature vector into an artificial intelligence model trained through artificial intelligence-based model training. In addition, the feature vector extraction unit may convert the neuromuscular system signal into a time-frequency domain for each of the divided plurality of segments, generate an envelope from the converted neuromuscular system signal for each of the divided plurality of segments, and analyze a pattern of the generated envelope to obtain a feature vector on a segment basis. In addition, the feature vector extraction unit may obtain an envelope peak using a positive peak and a negative peak, and generate an envelope by interpolating between the envelope peaks.

In addition, yet another embodiment for solving the above-described technical problems may provide a computer program stored on a computer-readable storage medium, wherein the computer program comprises instructions for causing one or more processors to perform neuromuscular system condition evaluation, the instructions comprising: obtaining a neuromuscular system signal; dividing the neuromuscular system signal into a plurality of segments based on frequency; extracting a feature vector for each of the divided plurality of segments; and performing neuromuscular system condition evaluation by inputting the extracted feature vector into an artificial intelligence model trained through artificial intelligence-based model training. In addition, the extracting of the feature vector may comprise: converting the neuromuscular system signal into a time-frequency domain for each of the divided plurality of segments; generating an envelope from the converted neuromuscular system signal for each of the divided plurality of segments; and analyzing a pattern of the generated envelope to obtain a feature vector on a segment basis. In addition, the generating of the envelope may comprise: obtaining an envelope peak using a positive peak and a negative peak; and generating an envelope by interpolating between the envelope peaks.

The embodiments described above will be described in detail below.

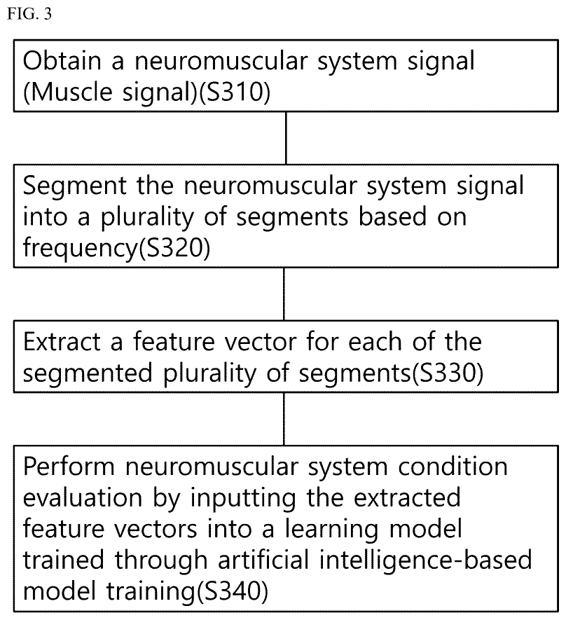

FIG. 3 is a diagram for describing a method of evaluating a neuromuscular system condition according to an embodiment of the present disclosure.

In step S310, the device (100) may acquire a neuromuscular system signal. In this case, the neuromuscular system signal may comprise the stimulated muscle contraction signal (SMCS) described above with reference to FIG. 1A and FIG. 1B.

The device (100) may apply multi-frequency electrical stimulation to a user, and as a result, the acquired stimulated muscle contraction signal (SMCS) may also include multiple frequencies.

In step S320, the device (100) may divide the acquired neuromuscular system signal into a plurality of segments based on frequency.

According to an embodiment of the present disclosure, the device (100) may apply electrical stimulation of different frequencies to a user over time. For example, the device (100) may apply electrical stimulation to a user at a frequency that increases by a predetermined frequency over time. As a specific example, the device (100) may apply electrical stimulation at a frequency of 5 Hz during a first time unit, electrical stimulation at a frequency of 10 Hz during a second time unit, and electrical stimulation at a frequency of 15 Hz during a third time unit.

In this case, the stimulated muscle contraction signal (SMCS) acquired by the device (100) may also include different frequencies over time. For example, the stimulated muscle contraction signal (SMCS) received in response to electrical stimulation at a frequency of 5 Hz may comprise a frequency of 5 Hz, the stimulated muscle contraction signal (SMCS) received in response to electrical stimulation at a frequency of 10 Hz may comprise a frequency of 10 Hz, and the stimulated muscle contraction signal (SMCS) received in response to electrical stimulation at a frequency of 15 Hz may comprise a frequency of 15 Hz.

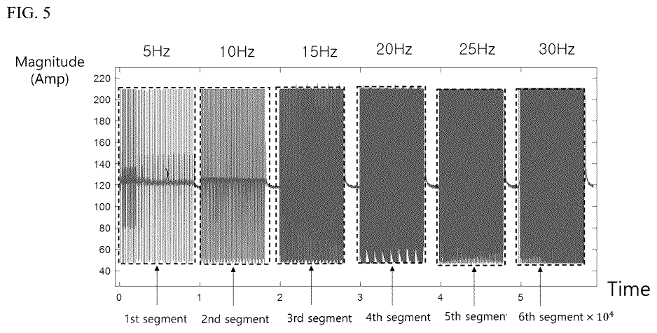

The device (100) may divide the acquired neuromuscular system signal into a plurality of segments based on frequency. For example, referring to FIG. 5, the device (100) may divide the acquired neuromuscular system signal into a plurality of segments. In this case, signals included in each segment may have the same frequency. In addition, in this case, signals included in each segment may be signals having frequencies within a specific range.

According to another embodiment of the present disclosure, the device (100) may acquire a neuromuscular system signal and analyze the frequencies included in the acquired neuromuscular system signal. In addition, the device (100) may divide the neuromuscular system signal into a plurality of segments by extracting signals for each frequency in predetermined units based on the analyzed frequencies.

In step S330, the device (100) may extract a feature vector for each of the divided plurality of segments.

According to an embodiment of the present disclosure, the device (100) may extract a feature vector for the neuromuscular system signal. In this case, the device (100) may extract a feature vector for each segment.

The device (100) may convert the neuromuscular system signal included in each of the plurality of segments into a time-frequency domain. According to an embodiment of the present disclosure, the neuromuscular system signal acquired by the device (100) is a signal in the time-magnitude domain, and the acquired neuromuscular system signal may be converted into the time-frequency domain for analysis. In this case, the device (100) may convert the signal into the time-frequency domain by performing a spectrogram transformation, but is not limited thereto, and may convert the acquired neuromuscular system signal into the time-frequency domain using various methods.

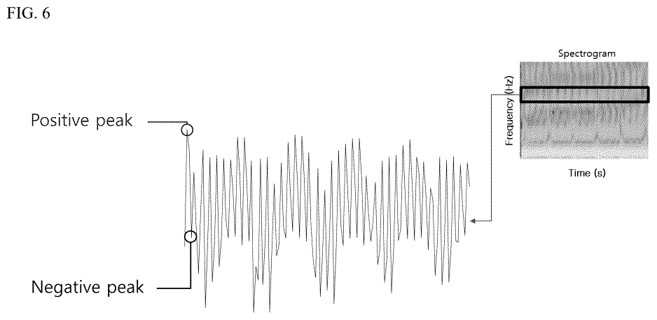

Referring to FIG. 6, the device (100) may obtain a positive peak and a negative peak from the neuromuscular system signal converted into the time-frequency domain. A positive peak refers to the peak at an inflection point where the signal transitions from rising to falling, and a negative peak refers to the peak at an inflection point where the signal transitions from falling to rising.

The device (100) may obtain an envelope peak based on at least one of the positive peak and the negative peak.

For example, the device (100) may obtain a positive peak as an envelope peak. In addition, the device (100) may obtain a negative peak as an envelope peak.

As another example, the device (100) may obtain an envelope peak by adding the magnitude values of adjacent positive and negative peaks.

As yet another example, the device (100) may obtain an envelope peak by calculating an average of the magnitude values of adjacent (e.g., neighboring) positive and negative peaks. As another example, the device (100) may obtain an envelope peak by calculating an average of the magnitude values of a plurality of adjacent positive peaks and a plurality of adjacent negative peaks.

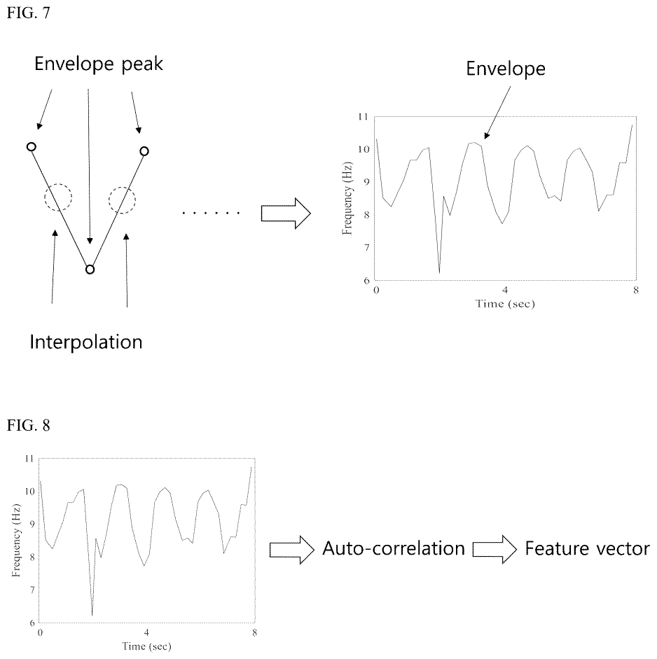

Referring to FIG. 7, the device (100) may obtain an envelope by interpolating between the envelope peaks. In this case, the device (100) may apply at least one of linear interpolation, parabolic interpolation, Newton interpolation, Lagrange interpolation, and spline interpolation, and is not limited thereto, and may interpolate the envelope peaks using various interpolation methods.

Referring to FIG. 8, the device (100) may extract a feature vector based on the generated envelope. In this case, the device (100) may obtain a feature vector by analyzing the pattern of the generated envelope.

In this case, the device (100) may extract a feature vector by performing at least one of sample entropy analysis, permutation entropy analysis, standard deviation signal analysis, square root analysis, auto-correlation analysis, cardinality analysis, and Poincaré plot analysis.

In step S340, the device (100) may perform neuromuscular system condition evaluation by inputting the extracted feature vector into an artificial intelligence model trained through artificial intelligence-based model training.

The device (100) may generate an artificial intelligence model by training on neuromuscular system signals.

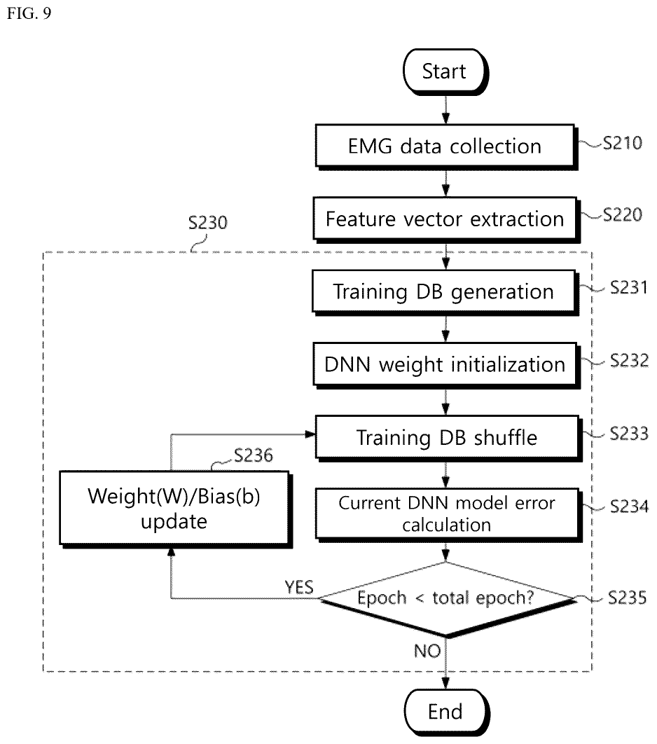

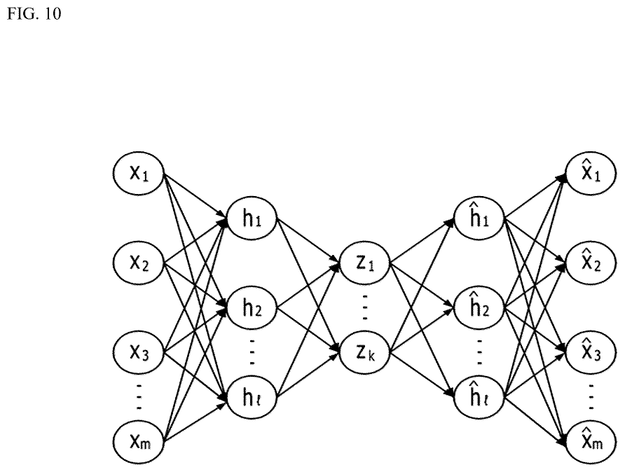

An exemplary method of generating an artificial intelligence model will be described in detail with reference to FIG. 9.

Referring to FIG. 9, in step S210, the device (100) may collect a neuromuscular system signal. The device (100) may apply electrical stimulation (ES) to body muscles and acquire a stimulated muscle contraction signal (SMCS). The stimulated muscle contraction signal (SMCS) may be a multi-frequency stimulated muscle contraction signal (SMCS) obtained by applying multi-frequency electrical stimulation to muscles.

In step S220, the device (100) may analyze the multi-frequency stimulated muscle contraction signal (SMCS) and extract a feature vector. The device (100) may remove noise electrical signals included in the multi-frequency stimulated muscle contraction signal (SMCS), and then extract a feature vector related to muscle strength, muscle endurance, and the like. In addition, the device (100) may perform artificial intelligence (AI) model training using the extracted feature vector.

In step S231, the device (100) may generate a training database (DB).

The device (100) may initialize deep neural network (DNN) weights (S232), shuffle the training database (DB) (S233), and calculate the current DNN model error (S234). The device (100) may determine whether the epoch trained so far is less than the last epoch (S235); if not less (NO), the process ends, and if less (YES), the device (100) may update the DNN weights and biases (S236) and perform step S233. Here, W denotes the weight parameter of the DNN, and b denotes the bias parameter of the DNN.

The device (100) is not limited thereto and may generate an artificial intelligence model in various ways.

According to an embodiment of the present disclosure, the device (100) may generate an artificial intelligence model for evaluating a neuromuscular system condition. For example, the device (100) may generate an artificial intelligence model for diagnosing sarcopenia. In addition, the device (100) may generate an artificial intelligence model for diagnosing muscle strength. In addition, the device (100) may generate an artificial intelligence model for evaluating muscle endurance.

In addition, according to another embodiment, the device (100) may generate an artificial intelligence model that performs at least one of muscle strength evaluation, muscle endurance evaluation, and sarcopenia diagnosis. For example, the device (100) may generate an artificial intelligence model that performs muscle strength evaluation and muscle endurance evaluation, may generate an artificial intelligence model that performs muscle endurance evaluation and sarcopenia diagnosis, and may generate an artificial intelligence model that performs sarcopenia diagnosis and muscle strength evaluation. In addition, the device (100) may generate an artificial intelligence model that performs at least one of muscle strength evaluation, muscle endurance evaluation, sarcopenia diagnosis, muscle fatigue (e.g., muscle capacity diagnosis, muscle degeneration state diagnosis, and muscle quality diagnosis), without being limited thereto.

The device (100) may perform muscle strength evaluation by inputting the extracted feature vector into the generated artificial intelligence model. For example, the device (100) may perform muscle strength evaluation by inputting the extracted feature vector into an artificial intelligence model for evaluating muscle strength. In addition, the device (100) may perform muscle endurance evaluation by inputting the extracted feature vector into an artificial intelligence model for evaluating muscle endurance. In addition, the device (100) may perform sarcopenia diagnosis by inputting the extracted feature vector into an artificial intelligence model for diagnosing sarcopenia.

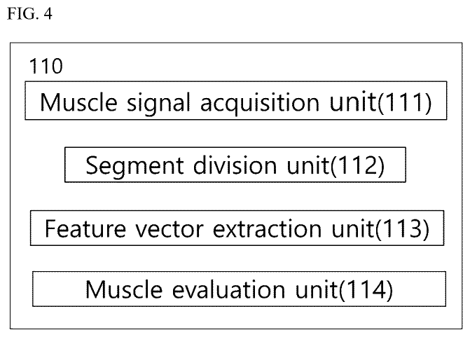

FIG. 4 is a diagram for describing functions of a processor according to an embodiment of the present disclosure.

The processor (110) may comprise at least one of a neuromuscular system signal acquisition unit (111), a segment division unit (112), a feature vector extraction unit (113), and a muscle evaluation unit (114).

The neuromuscular system signal acquisition unit (111) may acquire the neuromuscular system signal obtained by the electrical stimulation and measurement unit (170). The segment division unit (112) may divide the neuromuscular system signal into a plurality of segments based on frequency, and the feature vector extraction unit (113) may extract a feature vector for each of the divided plurality of segments. In addition, the muscle evaluation unit (114) may perform neuromuscular system condition evaluation by inputting the extracted feature vector into an artificial intelligence model trained through artificial intelligence-based model training. A detailed description thereof has been provided above with reference to FIG. 1A through FIG. 3.

FIG. 5 is a diagram for describing a method of dividing an acquired neuromuscular system signal into a plurality of segments according to an embodiment of the present disclosure.

FIG. 6 is a diagram for describing a method of generating an envelope for each of a plurality of segments according to an embodiment of the present disclosure.

FIG. 7 is a diagram for describing a method of applying interpolation to generate an envelope according to an embodiment of the present disclosure.

FIG. 8 is a diagram for describing a method of extracting a feature vector from an envelope according to an embodiment of the present disclosure.

FIG. 9 is a diagram for describing a method of generating an artificial intelligence model according to an embodiment of the present disclosure.

FIG. 5 through FIG. 9 have been described in detail in the description of FIG. 3 above.

FIG. 10 is a schematic diagram illustrating an artificial neural network according to an embodiment of the present disclosure.

Throughout the present specification, the terms “computational model,” “neural network,” “network function,” and “neural network” may be used interchangeably. A neural network may generally be composed of a set of interconnected computational units, which may be referred to as nodes. Such nodes may also be referred to as neurons. A neural network is configured to include at least one or more nodes. The nodes (or neurons) constituting a neural network may be interconnected by one or more links.

Within a neural network, one or more nodes connected through a link may form an input node and output node relationship with respect to each other. The concept of input nodes and output nodes is relative; any node that is in an output node relationship with respect to one node may be in an input node relationship with respect to another node, and vice versa. As described above, the input node-to-output node relationship may be formed around a link. One or more output nodes may be connected to one input node through links, and vice versa.

In the relationship between an input node and an output node connected through a single link, the value of the output node's data may be determined based on the data input to the input node. Here, the link interconnecting the input node and the output node may have a weight. The weight may be variable and may be varied by a user or an algorithm in order for the neural network to perform a desired function. For example, when one or more input nodes are interconnected to one output node by respective links, the output node may determine the output node value based on the values input to the input nodes connected to the output node and the weights set in the links corresponding to the respective input nodes.

As described above, in a neural network, one or more nodes are interconnected through one or more links to form input node and output node relationships within the neural network. The characteristics of the neural network may be determined according to the number of nodes and links constituting the neural network, the associations between the nodes and links, and the weight values assigned to each of the links. For example, if two neural networks exist with the same number of nodes and links but different weight values for the links, the two neural networks may be recognized as different from each other.

A neural network may be composed of a set of one or more nodes. A subset of the nodes constituting the neural network may constitute a layer. Some of the nodes constituting the neural network may constitute a single layer based on their distances from the initial input node. For example, a set of nodes at a distance of n from the initial input node may constitute layer n. The distance from the initial input node may be defined by the minimum number of links that must be traversed to reach the corresponding node from the initial input node. However, this definition of a layer is arbitrary for the purpose of explanation, and the order of layers within a neural network may be defined in a manner different from that described above. For example, a layer of nodes may be defined by the distance from the final output node.

The initial input node may refer to one or more nodes in the neural network to which data is directly input without passing through a link in relation to other nodes. Alternatively, within a neural network, in terms of the relationships between nodes based on links, it may refer to nodes that do not have other input nodes connected by links. Similarly, the final output node may refer to one or more nodes among the nodes in the neural network that do not have an output node in relation to other nodes. In addition, a hidden node may refer to nodes constituting the neural network that are neither the initial input node nor the final output node.

According to an embodiment of the present disclosure, a neural network may be one in which the number of nodes in the input layer is equal to the number of nodes in the output layer, and in which the number of nodes decreases and then increases again as the network progresses from the input layer to the hidden layers. According to another embodiment of the present disclosure, a neural network may be one in which the number of nodes in the input layer is less than the number of nodes in the output layer, and in which the number of nodes decreases as the network progresses from the input layer to the hidden layers. According to yet another embodiment of the present disclosure, a neural network may be one in which the number of nodes in the input layer is greater than the number of nodes in the output layer, and in which the number of nodes increases as the network progresses from the input layer to the hidden layers. According to yet another embodiment of the present disclosure, a neural network may be a combination of the neural networks described above.

A deep neural network (DNN) may refer to a neural network that includes a plurality of hidden layers in addition to an input layer and an output layer. Using a deep neural network, latent structures in data can be identified. That is, latent structures in photographs, text, video, audio, and music (e.g., what objects are in a photograph, what the content and sentiment of text are, what the content and sentiment of speech are, etc.) can be identified.

A deep neural network may comprise a convolutional neural network (CNN), a recurrent neural network (RNN), an autoencoder, a generative adversarial network (GAN), a restricted Boltzmann machine (RBM), a deep belief network (DBN), a Q-network, a U-network, a Siamese network, and a generative adversarial network (GAN), among others. The foregoing description of deep neural networks is illustrative only, and the present disclosure is not limited thereto.

In an embodiment of the present disclosure, the network function may comprise an autoencoder. An autoencoder may be a type of artificial neural network for outputting output data similar to input data. An autoencoder may comprise at least one hidden layer, and an odd number of hidden layers may be disposed between the input and output layers. The number of nodes in each layer may be reduced from the number of nodes in the input layer to an intermediate layer called a bottleneck layer (encoding), and then expanded symmetrically from the bottleneck layer to the output layer (symmetrical to the input layer). An autoencoder may perform nonlinear dimensionality reduction. The number of input and output layers may correspond to the dimensions after preprocessing of the input data. In an autoencoder structure, the number of nodes in the hidden layers included in the encoder may decrease as the distance from the input layer increases. The number of nodes in the bottleneck layer (the layer with the fewest nodes, located between the encoder and decoder) may be maintained above a certain number (e.g., at least half the number of nodes in the input layer) since insufficient information may be conveyed if the number is too small.

A neural network may be trained by at least one of supervised learning, unsupervised learning, semi-supervised learning, and reinforcement learning. Training of a neural network may be a process of applying to the neural network knowledge for the neural network to perform a specific operation.

A neural network may be trained in a direction that minimizes errors in the output. In the training of a neural network, training data is repeatedly input to the neural network, the error between the output of the neural network with respect to the training data and the target is calculated, and the error of the neural network is backpropagated from the output layer to the input layer of the neural network in a direction to reduce the error, thereby updating the connection weight of each node of the neural network. In the case of supervised learning, training data in which a correct answer is labeled for each piece of training data is used (i.e., labeled training data), while in the case of unsupervised learning, a correct answer may not be labeled for each piece of training data. That is, for example, training data for supervised learning on data classification may be data in which a category is labeled for each piece of training data. Labeled training data is input to the neural network, and an error may be calculated by comparing the output (category) of the neural network with the label of the training data. As another example, in the case of unsupervised learning on data classification, an error may be calculated by comparing the input training data with the neural network output. The calculated error is backpropagated in the reverse direction in the neural network (i.e., from the output layer toward the input layer), and the connection weights of the nodes in each layer of the neural network may be updated according to the backpropagation. The amount of change in the connection weight of each updated node may be determined according to a learning rate. The computation of the neural network with respect to input data and the backpropagation of errors may constitute a training cycle (epoch). The learning rate may be applied differently depending on the number of repetitions of the training cycle of the neural network. For example, a high learning rate may be used in the early stages of neural network training so that the neural network quickly achieves a certain level of performance, thereby improving efficiency, while a low learning rate may be used in the later stages of training to improve accuracy.

In the training of a neural network, training data is generally a subset of actual data (i.e., data to be processed using the trained neural network), and therefore, there may be a training cycle in which the error on the training data decreases but the error on the actual data increases. Overfitting is a phenomenon in which the neural network is excessively trained on the training data, resulting in increased error on actual data. For example, a phenomenon in which a neural network trained to recognize cats by being shown yellow cats fails to recognize cats of colors other than yellow may be a type of overfitting. Overfitting may act as a cause of increased errors in machine learning algorithms. Various optimization methods may be used to prevent such overfitting. To prevent overfitting, methods such as increasing training data, regularization, dropout (deactivating some nodes of the network during training), and the use of a batch normalization layer may be applied.

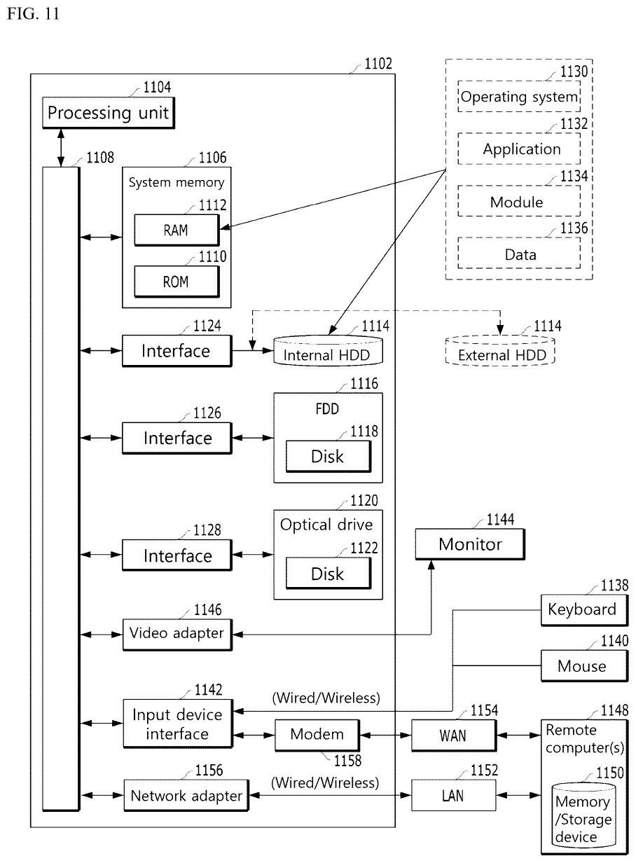

FIG. 11 is a general schematic diagram of an exemplary device (100) in which embodiments of the present disclosure may be implemented.

Although the present disclosure has been described above as being generally implementable by a computing device, those skilled in the art will appreciate that the present disclosure may be implemented in combination with computer-executable instructions and/or other program modules executable on one or more computers, and/or as a combination of hardware and software.

In general, program modules include routines, programs, components, data structures, and the like that perform particular tasks or implement particular abstract data types. Those skilled in the art will also appreciate that the methods of the present disclosure may be practiced with other computer system configurations, including single-processor or multiprocessor computer systems, minicomputers, mainframe computers, as well as personal computers, handheld computing devices, microprocessor-based or programmable consumer electronics, and the like, each of which may operate in connection with one or more associated devices.

The described embodiments of the present disclosure may also be practiced in distributed computing environments where certain tasks are performed by remote processing devices that are linked through a communications network. In a distributed computing environment, program modules may be located in both local and remote memory storage devices.

A computer typically includes a variety of computer-readable media. Any media accessible by a computer may be computer-readable media, and such computer-readable media includes volatile and nonvolatile media, transitory and non-transitory media, and removable and non-removable media. By way of example, and not limitation, computer-readable media may comprise computer-readable storage media and computer-readable transmission media. Computer-readable storage media includes volatile and nonvolatile media, transitory and non-transitory media, and removable and non-removable media implemented in any method or technology for storage of information such as computer-readable instructions, data structures, program modules, or other data. Computer-readable storage media includes, but is not limited to, RAM, ROM, EEPROM, flash memory or other memory technology, CD-ROM, digital video disk (DVD) or other optical disk storage, magnetic cassettes, magnetic tape, magnetic disk storage or other magnetic storage devices, or any other medium that can be accessed by a computer and used to store desired information.

Computer-readable transmission media typically embodies computer-readable instructions, data structures, program modules, or other data in a modulated data signal such as a carrier wave or other transport mechanism, and includes all information delivery media. The term “modulated data signal” means a signal that has one or more of its characteristics set or changed in such a manner as to encode information in the signal. By way of example, and not limitation, computer-readable transmission media includes wired media such as a wired network or direct-wired connection, and wireless media such as acoustic, RF, infrared, and other wireless media. Combinations of any of the above-described media are also included within the scope of computer-readable transmission media.

An exemplary environment (1100) implementing various aspects of the present disclosure including a computer (1102) is shown, wherein the computer (1102) includes a processing unit (1104), a system memory (1106), and a system bus (1108). The system bus (1108) couples system components including, but not limited to, the system memory (1106) to the processing unit (1104). The processing unit (1104) may be any of various commercially available processors. Dual processor and other multiprocessor architectures may also be used as the processing unit (1104).

The system bus (1108) may be any of several types of bus structures that may further interconnect to a memory bus, a peripheral bus, and a local bus using any of a variety of commercially available bus architectures. The system memory (1106) includes read-only memory (ROM) (1110) and random access memory (RAM) (1112). A basic input/output system (BIOS) is stored in non-volatile memory (1110) such as ROM, EPROM, and EEPROM, and this BIOS contains basic routines that help transfer information between components within the computer (1102), such as during startup. The RAM (1112) may also include high-speed RAM such as static RAM for caching data.

The computer (1102) also includes an internal hard disk drive (HDD) (1114) (e.g., EIDE, SATA)—which may also be configured for external use in a suitable chassis (not shown)—a magnetic floppy disk drive (FDD) (1116) (e.g., for reading from or writing to a removable diskette (1118)), and an optical disk drive (1120) (e.g., for reading a CD-ROM disk (1122) or for reading from or writing to other high-capacity optical media such as a DVD). The hard disk drive (1114), magnetic disk drive (1116), and optical disk drive (1120) may be connected to the system bus (1108) by a hard disk drive interface (1124), a magnetic disk drive interface (1126), and an optical drive interface (1128), respectively. The interface (1124) for external drive implementations includes at least one or both of Universal Serial Bus (USB) and IEEE 1394 interface technologies.

These drives and their associated computer-readable media provide non-volatile storage of data, data structures, computer-executable instructions, and the like. In the case of the computer (1102), the drives and media correspond to the storage of any data in a suitable digital format. Although the foregoing description of computer-readable media refers to an HDD, removable magnetic disks, and removable optical media such as CDs or DVDs, those skilled in the art will appreciate that other types of media readable by a computer, such as zip drives, magnetic cassettes, flash memory cards, cartridges, and the like, may also be used in the exemplary operating environment, and that any such media may contain computer-executable instructions for performing the methods of the present disclosure.

A number of program modules may be stored in the drives and RAM (1112), including an operating system (1130), one or more application programs (1132), other program modules (1134), and program data (1136). All or portions of the operating system, applications, modules, and/or data may also be cached in RAM (1112). It will be appreciated that the present disclosure may be implemented in various commercially available operating systems or combinations of operating systems.

A user may enter commands and information into the computer (1102) through one or more wired/wireless input devices, for example, a keyboard (1138) and a pointing device such as a mouse (1140). Other input devices (not shown) may comprise a microphone, an IR remote control, a joystick, a game pad, a stylus pen, a touch screen, and the like. These and other input devices are often connected to the processing unit (1104) through an input device interface (1142) connected to the system bus (1108), but may be connected by other interfaces such as a parallel port, an IEEE 1394 serial port, a game port, a USB port, an IR interface, and the like.

A monitor (1144) or other type of display device is also connected to the system bus (1108) via an interface such as a video adapter (1146). In addition to the monitor (1144), a computer generally includes other peripheral output devices (not shown) such as speakers and a printer.

The computer (1102) may operate in a networked environment using logical connections to one or more remote computers, such as remote computer(s) (1148), via wired and/or wireless communication. The remote computer(s) (1148) may be a workstation, a computing device computer, a router, a personal computer, a portable computer, a microprocessor-based entertainment device, a peer device, or other common network node, and generally includes many or all of the components described for the computer (1102), but for simplicity, only a memory storage device (1150) is shown. The logical connections depicted include wired/wireless connections to a local area network (LAN) (1152) and/or a larger network, such as a wide area network (WAN) (1154). Such LAN and WAN networking environments are commonplace in offices and companies, and facilitate enterprise-wide computer networks such as intranets, all of which may be connected to a worldwide computer network such as the Internet.

When used in a LAN networking environment, the computer (1102) is connected to the local network (1152) through a wired and/or wireless communication network interface or adapter (1156). The adapter (1156) may facilitate wired or wireless communication to the LAN (1152), which also includes a wireless access point installed thereon for communicating with the wireless adapter (1156). When used in a WAN networking environment, the computer (1102) may comprise a modem (1158), or may be connected to a communications computing device on the WAN (1154), or may have other means of establishing communications over the WAN (1154), such as over the Internet. The modem (1158), which may be internal or external and a wired or wireless device, is connected to the system bus (1108) through the serial port interface (1142). In a networked environment, program modules described with respect to the computer (1102), or portions thereof, may be stored in the remote memory/storage device (1150). It will be appreciated that the network connections shown are exemplary and other means of establishing a communications link between the computers may be used.

The computer (1102) is operable to communicate with any wireless devices or entities deployed in wireless communication, for example, printers, scanners, desktop and/or portable computers, portable data assistants (PDAs), communications satellites, any equipment or locations associated with wirelessly detectable tags, and telephones. This includes at least Wi-Fi and Bluetooth wireless technologies. Thus, the communication may be a predefined structure as with a conventional network, or simply an ad hoc communication between at least two devices.

Wi-Fi (Wireless Fidelity) enables connection to the Internet and the like without wires. Wi-Fi is a wireless technology, like cell phones, that enables such devices, e.g., computers, to send and receive data indoors and outdoors, anywhere within the coverage area of a base station. Wi-Fi networks use a wireless technology called IEEE 802.11 (a, b, g, etc.) to provide secure, reliable, and high-speed wireless connections. Wi-Fi may be used to connect computers to each other, to the Internet, and to wired networks (using IEEE 802.3 or Ethernet). Wi-Fi networks operate in unlicensed 2.4 and 5 GHz radio bands, for example, at 11 Mbps (802.11a) or 54 Mbps (802.11b) data rates, or in products that include both bands (dual band).

An embodiment for solving one of the technical problems of the present disclosure may provide a method and device for analyzing a neuromuscular system signal generated in muscles. In particular, when muscles are stimulated by electrical stimulation, involuntary muscle contraction also occurs, and an analysis technology for neuromuscular system technology may be provided to distinguish between involuntary muscle contraction and voluntary muscle contraction.

One embodiment of the present disclosure for solving the above-described problems may provide a method, performed by a device, of performing neuromuscular system signal analysis, the method comprising: obtaining a neuromuscular system signal; time-dividing the neuromuscular system signal into a plurality of neuromuscular system signal frames; and performing linear predictive coding (LPC) on each of the neuromuscular system signal frames to remove a noise signal and extract a voluntary muscle contraction signal.

In addition, the neuromuscular system signal may comprise at least one of a voluntary muscle contraction signal, an involuntary muscle contraction signal, and a muscle treatment signal.

In addition, the time-dividing of the neuromuscular system signal into a plurality of neuromuscular system signal frames may be performed on a neuromuscular system signal acquired after a time point at which a muscle treatment signal is applied to a muscle or a time point at which voluntary muscle contraction occurs.

In addition, the performing of linear predictive coding (LPC) on each of the neuromuscular system signal frames to remove a noise signal and extract a voluntary muscle contraction signal may comprise: performing frequency filtering on each of the neuromuscular system signal frames to generate a frequency-filtered neuromuscular system signal.

In addition, the performing of linear predictive coding (LPC) on each of the neuromuscular system signal frames to remove a noise signal and extract a voluntary muscle contraction signal may further comprise: performing a fast Fourier transform (FFT) on the frequency-filtered neuromuscular system signal to generate an FFT neuromuscular system signal.

In addition, the performing of linear predictive coding (LPC) on each of the neuromuscular system signal frames to remove a noise signal and extract a voluntary muscle contraction signal may further comprise: performing linear predictive coding (LPC) on the frequency-filtered neuromuscular system signal to generate an LPC neuromuscular system signal.

In addition, the performing of linear predictive coding (LPC) on each of the neuromuscular system signal frames to remove a noise signal and extract a voluntary muscle contraction signal may further comprise: computing the FFT neuromuscular system signal and the LPC neuromuscular system signal to generate an encoded neuromuscular system signal.

In addition, the performing of linear predictive coding (LPC) on each of the neuromuscular system signal frames to remove a noise signal and extract a voluntary muscle contraction signal may further comprise: generating a residual signal between the encoded neuromuscular system signal and a buffer signal.

In addition, the buffer signal may comprise a signal composed solely of noise, and may be updated based on patient information and predetermined conditions.

In addition, the buffer signal may comprise at least one of: a neuromuscular system signal acquired before electrical stimulation treatment, a neuromuscular system signal acquired during a previous treatment, a neuromuscular system signal acquired after completion of a previous treatment, a neuromuscular system signal of a previous frame, an overlapped signal of a predetermined number of previous frames' neuromuscular system signals, and a reference point neuromuscular system signal.

In addition, the performing of linear predictive coding (LPC) on each of the neuromuscular system signal frames to remove a noise signal and extract a voluntary muscle contraction signal may further comprise: applying an inverse fast Fourier transform (IFFT) to the result of computing the LPC neuromuscular system signal and the residual signal to extract the voluntary muscle contraction signal to be analyzed.

In addition, another embodiment of the present disclosure for solving the above-described problems may provide a device for performing neuromuscular system signal analysis, the device comprising: a neuromuscular system signal acquisition unit configured to acquire a neuromuscular system signal; a neuromuscular system signal time-division unit configured to time-divide the neuromuscular system signal into a plurality of neuromuscular system signal frames; and a neuromuscular system signal extraction unit configured to perform linear predictive coding on each of the neuromuscular system signal frames to remove a noise signal and extract a voluntary muscle contraction signal.

In addition, the neuromuscular system signal extraction unit may perform frequency filtering on each of the neuromuscular system signal frames to generate a frequency-filtered neuromuscular system signal.

In addition, the neuromuscular system signal extraction unit may additionally perform a fast Fourier transform (FFT) on the frequency-filtered neuromuscular system signal to generate an FFT neuromuscular system signal.

In addition, the neuromuscular system signal extraction unit may additionally perform linear predictive coding (LPC) on the frequency-filtered neuromuscular system signal to generate an LPC neuromuscular system signal.

In addition, the neuromuscular system signal extraction unit may additionally compute the FFT neuromuscular system signal and the LPC neuromuscular system signal to generate an encoded neuromuscular system signal.

In addition, the neuromuscular system signal extraction unit may additionally generate a residual signal between the encoded neuromuscular system signal and a buffer signal.

In addition, the neuromuscular system signal extraction unit may additionally apply an inverse fast Fourier transform (IFFT) to the result of computing the LPC neuromuscular system signal and the residual signal to extract the voluntary muscle contraction signal to be analyzed.

In addition, yet another embodiment of the present disclosure for solving the above-described problems may provide a computer program stored on a computer-readable storage medium, wherein the computer program comprises instructions for causing one or more processors to perform neuromuscular system signal analysis, the instructions comprising: obtaining a neuromuscular system signal; time-dividing the neuromuscular system signal into a plurality of neuromuscular system signal frames; and performing linear predictive coding on each of the neuromuscular system signal frames to remove a noise signal and extract a voluntary muscle contraction signal.

In addition, the performing of linear predictive coding on each of the neuromuscular system signal frames to remove a noise signal and extract a voluntary muscle contraction signal may comprise: performing frequency filtering on each of the neuromuscular system signal frames to generate a frequency-filtered neuromuscular system signal.

In addition, the performing of linear predictive coding on each of the neuromuscular system signal frames to remove a noise signal and extract a voluntary muscle contraction signal may further comprise: performing a fast Fourier transform (FFT) on the frequency-filtered neuromuscular system signal to generate an FFT neuromuscular system signal.

In addition, the performing of linear predictive coding on each of the neuromuscular system signal frames to remove a noise signal and extract a voluntary muscle contraction signal may further comprise: performing linear predictive coding (LPC) on the frequency-filtered neuromuscular system signal to generate an LPC neuromuscular system signal.

In addition, the performing of linear predictive coding on each of the neuromuscular system signal frames to remove a noise signal and extract a voluntary muscle contraction signal may further comprise: computing the FFT neuromuscular system signal and the LPC neuromuscular system signal to generate an encoded neuromuscular system signal.

In addition, the performing of linear predictive coding on each of the neuromuscular system signal frames to remove a noise signal and extract a voluntary muscle contraction signal may further comprise: generating a residual signal between the encoded neuromuscular system signal and a buffer signal.

In addition, the performing of linear predictive coding on each of the neuromuscular system signal frames to remove a noise signal and extract a voluntary muscle contraction signal may further comprise: applying an inverse fast Fourier transform (IFFT) to the result of computing the LPC neuromuscular system signal and the residual signal to extract the voluntary muscle contraction signal to be analyzed.

The embodiments for solving the above-described technical problems will be described in detail below.

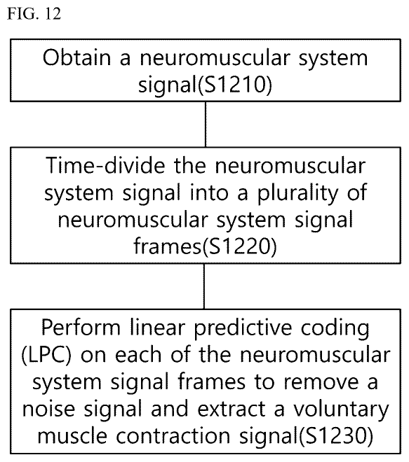

FIG. 12 is a diagram for describing a method of obtaining a neuromuscular system signal according to an embodiment of the present disclosure.

In step S1210, the device (100) may acquire a neuromuscular system signal. As described above, the neuromuscular system signal may comprise a voluntary muscle contraction signal to be analyzed, an involuntary muscle contraction signal as noise, a muscle treatment signal, and other noise.

In step S1220, the device (100) may time-divide the acquired neuromuscular system signal into a plurality of neuromuscular system signal frames. For example, each of the neuromuscular system signal frames may be a neuromuscular system signal measured over 600 ms; however, the foregoing frame length is illustrative only, and the present disclosure is not limited thereto. The device (100) may divide the signal into frame units in order to analyze the neuromuscular system signal in the frequency domain or to remove noise. Since the neuromuscular system signal has time-varying characteristics in which signal properties may change over time, the device (100) may time-divide the neuromuscular system signal into neuromuscular system signal frames for analysis.

In addition, the device (100) may perform time-division of the neuromuscular system signal with respect to a neuromuscular system signal acquired after the time point at which a muscle treatment signal is applied to a muscle or the time point at which voluntary muscle contraction occurs. When a patient voluntarily moves their muscles to generate a voluntary muscle contraction signal, the device (100) may collect and analyze the neuromuscular system signal from that time point in order to acquire such voluntary muscle contraction signal.

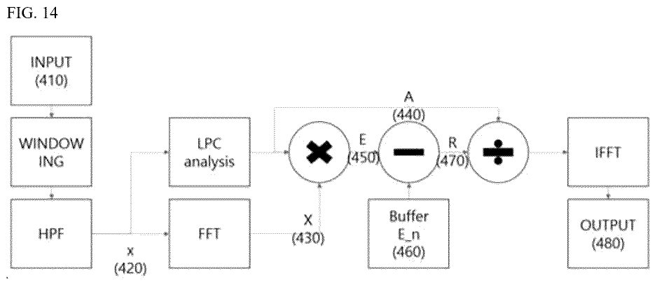

In step S1230, the device (100) may remove noise signals from each of the neuromuscular system signal frames to extract a voluntary muscle contraction signal. As described above, the noise signal may comprise an involuntary muscle contraction signal, a muscle treatment signal, and other noise. The process of removing noise from the neuromuscular system signal will be described in detail below with reference to FIG. 14.

The device (100) may perform frequency filtering on each of the neuromuscular system signal frames (410) to generate a frequency-filtered neuromuscular system signal (x, 420). The frequency filtering may be, for example, high pass filtering (HPF) that filters out signals below a predetermined frequency. Through this, the device (100) may remove measurement noise and the like from each of the neuromuscular system signal frames (410).

The device (100) may perform a fast Fourier transform (FFT) on the frequency-filtered neuromuscular system signal (420) to generate an FFT neuromuscular system signal (X, 430). The device (100) may perform a Fourier transform on the frequency-filtered neuromuscular system signal (420) in order to analyze the signal in the frequency domain.

The device (100) may perform linear predictive coding (LPC) on the frequency-filtered neuromuscular system signal (420) to generate an LPC neuromuscular system signal (A, 440). The device (100) may extract characteristics of the neuromuscular system signal generation model and perform compression of the neuromuscular system signal through linear predictive coding. The device (100) may generate the LPC neuromuscular system signal (440) by predicting the neuromuscular system signal of the current frame as an approximate linear combination of the neuromuscular system signals of previous frames and encoding the differential component.

The device (100) may compute the FFT neuromuscular system signal (430) and the LPC neuromuscular system signal (440) to generate an encoded neuromuscular system signal (E, 450). The computation of the FFT neuromuscular system signal (430) and the LPC neuromuscular system signal (440) may be, for example, a multiplication operation in the frequency domain.

The device (100) may generate a residual signal (R, 470) between the encoded neuromuscular system signal (450) and a buffer signal (E_n, 460). The device (100) may generate the residual signal (470) through a subtraction operation between the encoded neuromuscular system signal (450) and the buffer signal (460).

The buffer signal is a signal pre-stored in a buffer, includes a signal composed solely of noise, and may be updated according to patient information and predetermined conditions. The muscle response to electrical stimulation may differ among patients, and accordingly, the muscle treatment signal applied may also differ. Therefore, the buffer signal stored in the buffer may be changed to match the patient currently being measured according to patient information. In addition, when electrical stimulation treatment for muscles is repeated, the muscle condition may improve through the treatment effect, and in this case, the noise signal may also change. Accordingly, the buffer signal may be newly measured and updated at regular intervals, such as the repetition of treatment sessions or the passage of time. For example, it may be updated for each patient at every treatment session or at every application of a treatment signal.

The buffer signal is a reference signal used to obtain the residual signal for noise removal, and may comprise at least one of: a neuromuscular system signal acquired before electrical stimulation treatment, a neuromuscular system signal acquired during a previous treatment, a neuromuscular system signal acquired after completion of a previous treatment, a neuromuscular system signal of a previous frame, an overlapped signal of a predetermined number of previous frames' neuromuscular system signals, and a reference point neuromuscular system signal. The neuromuscular system signal acquired before electrical stimulation treatment may be the neuromuscular system signal measured for the first time for a patient, serving as a reference signal before a treatment signal is applied. The neuromuscular system signal acquired during a previous treatment may comprise the neuromuscular system signal obtained during a previous electrical stimulation treatment session. The neuromuscular system signal acquired after completion of a previous treatment may comprise the neuromuscular system signal obtained after a previous electrical stimulation treatment is completed. The neuromuscular system signal of a previous frame may comprise the neuromuscular system signal obtained in the previous frame. The overlapped signal of a predetermined number of previous frames' neuromuscular system signals may be an average value of the signals of previous frames, or a signal extracted by taking only the overlapping signals among the neuromuscular system signals of the predetermined number of previous frames. The reference point neuromuscular system signal may be obtained by separately measuring a neuromuscular system signal that serves as a reference for the noise signal for the purpose of measuring treatment effects.

The device (100) may apply an inverse fast Fourier transform (IFFT) to the result of computing the LPC neuromuscular system signal (440) and the residual signal (470) to extract the voluntary muscle contraction signal to be analyzed. The computation of the LPC neuromuscular system signal (440) and the residual signal (470) may be, for example, a division operation in which the LPC neuromuscular system signal (440) is divided by the residual signal (470).

In an additional step, the device (100) may analyze the muscle condition based on the voluntary muscle contraction signal, and may determine at least one of a muscle treatment effect and a muscle treatment signal based on the muscle condition. The device (100) may evaluate the muscle condition based on the obtained voluntary muscle contraction signal to analyze whether the treatment signal is effective and whether there is a treatment effect from the treatment signal. Accordingly, the device (100) may determine whether to apply a muscle treatment signal and whether to change the muscle treatment signal.

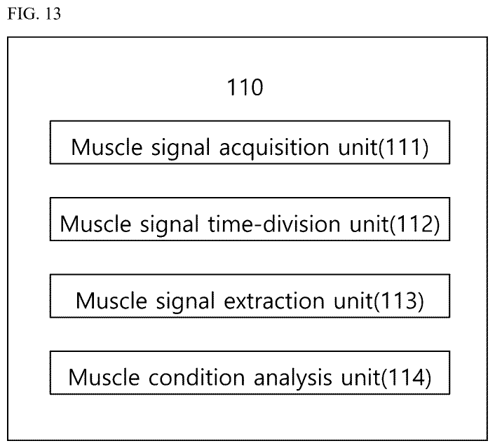

FIG. 13 is a diagram for describing functions of a processor according to an embodiment of the present disclosure.

The processor (110) may comprise at least one of a neuromuscular system signal acquisition unit (111), a neuromuscular system signal time-division unit (112), a neuromuscular system signal extraction unit (113), and a muscle condition analysis unit (114).

The neuromuscular system signal acquisition unit (111) may acquire the neuromuscular system signal obtained by the electrical stimulation and measurement unit (170). The neuromuscular system signal time-division unit (112) may time-divide the neuromuscular system signal into a plurality of neuromuscular system signal frames. The neuromuscular system signal extraction unit (113) may remove noise from each of the neuromuscular system signal frames to extract a voluntary muscle contraction signal. In addition, the muscle condition analysis unit (114) may evaluate the muscle condition using an artificial intelligence model trained through artificial intelligence-based model training, and may determine a muscle treatment effect and a muscle treatment signal for muscle treatment based on the muscle condition. A detailed description thereof has been provided above.

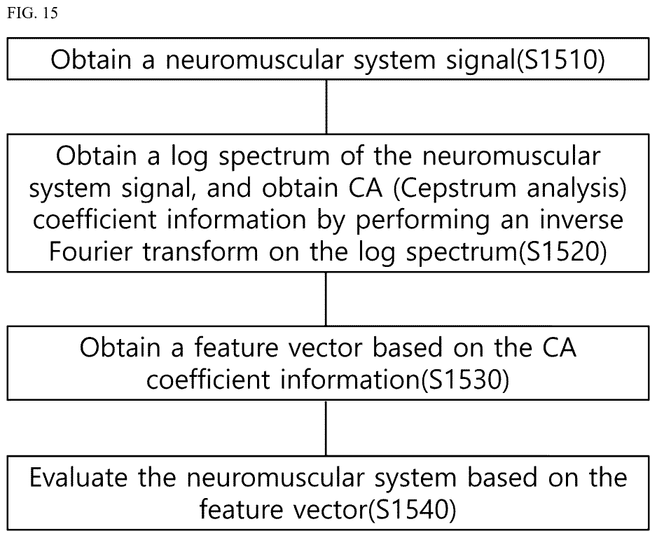

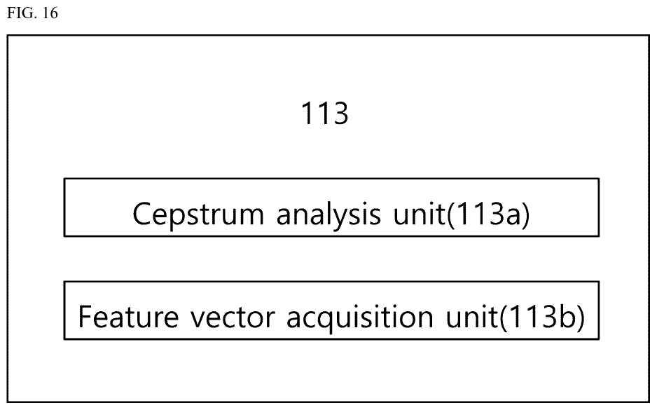

FIG. 15 is a diagram for describing a method of evaluating a neuromuscular system according to another embodiment of the present disclosure.

In step S1510, the device (100) may acquire a neuromuscular system signal. In this case, the neuromuscular system signal may comprise the stimulated muscle contraction signal (SMCS) described above with reference to FIG. 1A and FIG. 1B.

The device (100) may apply multi-frequency electrical stimulation to a user. In addition, a signal may be acquired from the neuromuscular system in response thereto. The acquired stimulated muscle contraction signal (SMCS) may comprise multiple frequencies.

In step S1520, the device (100) may obtain a log spectrum of the neuromuscular system signal and perform an Inverse Fourier Transform on the log spectrum to obtain CA (cepstrum analysis) coefficient information.

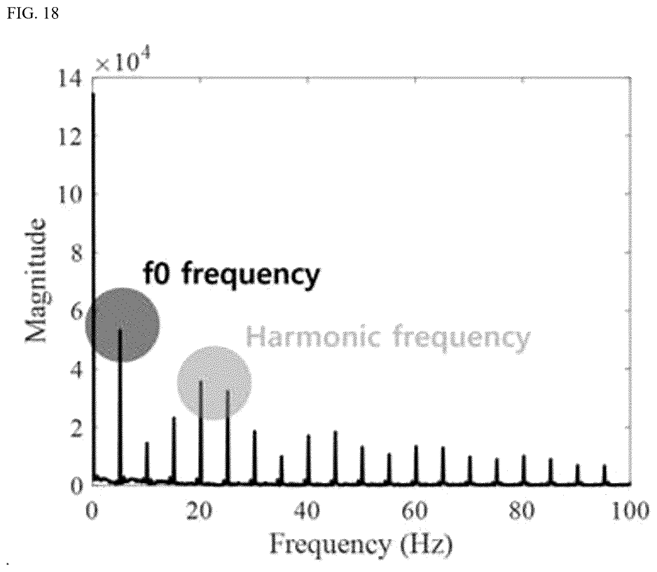

According to an embodiment of the present disclosure, the device (100) may perform a Fourier Transform on the neuromuscular system signal to generate FT (Fourier Transform) neuromuscular system signal information (see FIG. 17).

According to an embodiment of the present disclosure, the device (100) may calculate a log spectrum of the neuromuscular system signal by applying a logarithmic function to the FT neuromuscular system signal information.

In addition, the device (100) may perform an Inverse Fourier Transform on the calculated log spectrum to obtain CA (cepstrum analysis) coefficient information (see FIG. 19).

In this case, the device (100) may obtain CA coefficient information by utilizing a complex value. In addition, the device (100) may obtain CA coefficient information by utilizing a real value. In addition, the device (100) may obtain CA coefficient information by squaring each of a complex value and a real value, adding the squared values, and utilizing the value obtained through a square root operation.

Specifically, the device (100) may obtain CA coefficient information by extracting only the complex value from the value obtained by performing an Inverse Fourier Transform on the calculated log spectrum. In addition, the device (100) may obtain CA coefficient information by extracting only the real value from the value obtained by performing an Inverse Fourier Transform on the calculated log spectrum. In addition, the device (100) may obtain CA coefficient information by extracting a complex value and a real value from the value obtained by performing an Inverse Fourier Transform on the calculated log spectrum, squaring each of the extracted complex value and the extracted real value, adding the squared values, and performing a square root operation (V {(complex value)× (complex value)+ (real value) x (real value)}). As a result, the device (100) may utilize a complex value as an input to the artificial intelligence model, may utilize a real value as an input to the artificial intelligence model, and may also utilize a value calculated through a square root operation as an input to the artificial intelligence model.

The obtained CA coefficient information may be classified into an FF (fundamental frequency) component, an HF (harmonic frequency) component, and other components.

In step S1530, the device (100) may obtain a feature vector based on the CA (cepstrum analysis) coefficient information.

According to an embodiment of the present disclosure, the device (100) may obtain a feature vector based on the obtained CA (cepstrum analysis) coefficient information. In addition, the device (100) may perform preprocessing on the obtained CA (cepstrum analysis) coefficient information and obtain a feature vector based on the preprocessed CA (cepstrum analysis) coefficient information.

The device (100) may perform preprocessing on the obtained CA (cepstrum analysis) coefficient information.

For example, the device (100) may generate preprocessed CA coefficient information by removing the FF (fundamental frequency) component generated by the electrical stimulation. In addition, the device (100) may generate preprocessed CA coefficient information by removing the HF (harmonic frequency) component generated by the electrical stimulation from the CA coefficient information. In addition, the device (100) may generate preprocessed CA coefficient information by removing both the FF (fundamental frequency) component and the HF (harmonic frequency) component generated by the electrical stimulation from the CA coefficient information.

According to an embodiment of the present disclosure, the FF (fundamental frequency) component may comprise information about the periodicity of the signal. For example, when there is a sine waveform that oscillates 5 times per second, the FF (fundamental frequency) component appears at 5 Hz.

According to an embodiment of the present disclosure, the HF (harmonic frequency) component may comprise nonlinear characteristics. For example, when the FF (fundamental frequency) component is 5 Hz, the HF (harmonic frequency) component may occur at 10 Hz, 15 Hz, 20 Hz, . . . 5×n Hz, and so on. As another example, when the FF (fundamental frequency) component is 10 Hz, the HF (harmonic frequency) component may occur at 20 Hz, 30 Hz, 40 Hz, . . . 10×n Hz, and so on.

According to an embodiment of the present disclosure, the device (100) may obtain a feature vector based on the obtained CA coefficient information. In addition, the device (100) may obtain a feature vector based on the preprocessed CA coefficient information.

In this case, the device (100) may obtain temporal change statistics information of the CA coefficient information (the obtained CA coefficient information and/or the preprocessed CA coefficient information), and obtain a feature vector based on the obtained temporal change statistics information. Specifically, the device (100) may obtain a feature vector based on at least one of a maximum value, a minimum value, a mean value, a median value, a standard deviation value, a variance value, and an IQR (inter-quartile range) value of the CA coefficient information (the obtained CA coefficient information and/or the preprocessed CA coefficient information) over time.

Specifically, the device (100) may acquire a stimulated muscle contraction signal (SMCS) over a predetermined time interval (e.g., 8 seconds) and divide the acquired stimulated muscle contraction signal into a plurality of predetermined sub-time intervals (e.g., 1 second). In addition, the device (100) may obtain CA coefficient information for each of the sub-time intervals based on the method described above, thereby obtaining candidate feature vectors for each of the sub-time intervals. In addition, the device (100) may obtain temporal change statistics information of the candidate feature vectors obtained in each of the sub-time intervals, and obtain a feature vector based on the obtained temporal change statistics information.

In step S1540, the device (100) may evaluate the neuromuscular system based on the feature vector.

According to an embodiment of the present disclosure, the device (100) may perform neuromuscular system evaluation by inputting the extracted feature vector into an artificial intelligence model trained through artificial intelligence-based model training. For example, the device (100) may perform at least one of sarcopenia diagnosis, muscle strength evaluation, muscle endurance evaluation, gait speed evaluation, and muscle mass evaluation by inputting the feature vector into an artificial intelligence model that evaluates at least one of sarcopenia diagnosis, muscle strength evaluation, muscle endurance evaluation, gait speed evaluation, and muscle mass evaluation.

An exemplary method of generating an artificial intelligence model has been described above.

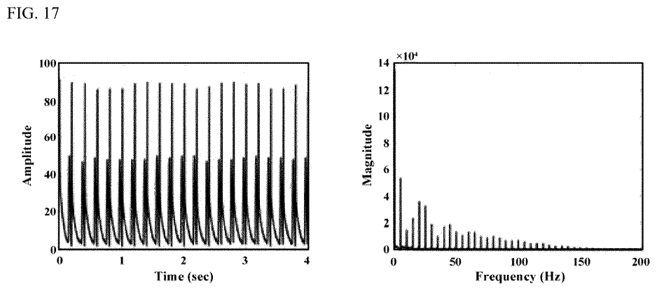

FIG. 16 is a diagram for describing functions of a processor according to an embodiment of the present disclosure.

The processor (110) may comprise at least one of a neuromuscular system signal acquisition unit (111), a segment division unit (112), a feature vector extraction unit (113), and a muscle evaluation unit (114). In addition, the feature vector extraction unit (113) may comprise at least one of a cepstrum analysis unit (113a) and a feature vector acquisition unit (113b). According to another embodiment of the present disclosure, the cepstrum analysis unit (113a) and the feature vector acquisition unit (113b) may not be included in the feature vector extraction unit (113).

The cepstrum analysis unit (113a) may perform a Fourier Transform on the neuromuscular system signal to generate FT (Fourier Transform) neuromuscular system signal information, and calculate a log spectrum of the neuromuscular system signal by applying a logarithmic function to the FT (Fourier Transform) neuromuscular system signal information. In addition, the cepstrum analysis unit (113a) may perform an Inverse Fourier Transform on the calculated log spectrum to obtain CA (cepstrum analysis) coefficient information. In addition, the feature vector acquisition unit (113b) may obtain a feature vector based on the CA coefficient information. In addition, the muscle condition evaluation unit (114) may evaluate the neuromuscular system based on the obtained feature vector. A detailed description thereof has been provided above.

FIGS. 17, 18, and 19 are reference diagrams for explaining signal information analysis according to an embodiment of the present disclosure.

FIG. 17 is a diagram illustrating that a stimulated muscle contraction signal (SMCS) included in a neuromuscular system signal has strongly periodic characteristics.

Also, FIG. 17 and FIG. 18 are exemplary diagrams in which a Fourier Transform is performed on the signal of FIG. 17 to generate FT neuromuscular system signal information, and are diagrams for confirming that an FF (fundamental frequency) component (f0) and HF (harmonic frequency) components are prominently visible.

Also, FIG. 19 is an exemplary diagram of CA (cepstrum analysis) coefficient information acquired by the device (100). The CA (cepstrum analysis) coefficient information acquired by the device (100) may comprise an FF (fundamental frequency) component and HF (harmonic frequency) components (1611), and may comprise a component (1612) excluding the FF (fundamental frequency) component and HF (harmonic frequency) components (1611).

The device (100) may obtain preprocessed CA coefficient information by removing the FF (fundamental frequency) component and HF (harmonic frequency) components (1611) from the CA (cepstrum analysis) coefficient information.

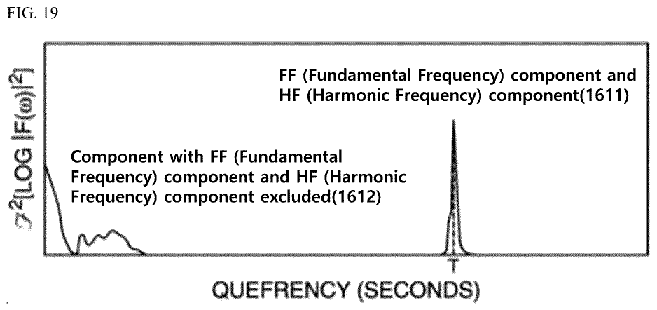

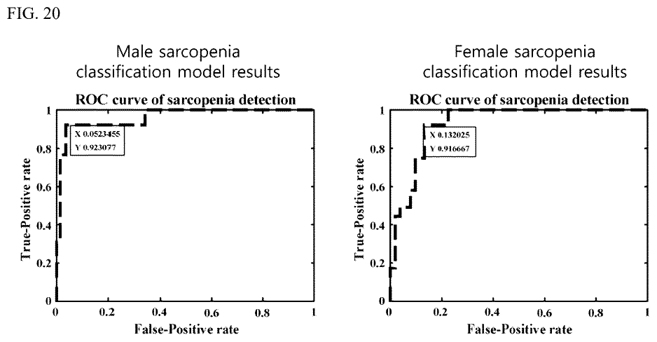

FIG. 20 is a diagram for explaining an experimental case according to an embodiment of the present disclosure.

According to an embodiment of the present disclosure, the experimental results confirmed that the male sarcopenia classification model and the female sarcopenia classification model each demonstrated excellent performance.

Specifically, according to an embodiment of the present disclosure, the performance of the male sarcopenia model showed a sensitivity of 92.3% and a specificity of 94.5%, and the performance of the female sarcopenia model showed a sensitivity of 91.7% and a specificity of 86.8%.

An embodiment for solving one of the technical problems of the present disclosure may provide a method and apparatus for evaluating a neuromuscular system by analyzing a neuromuscular system signal based on electrical stimulation (ES).

An embodiment of the present disclosure for solving the foregoing problems may provide a method by which a device evaluates a neuromuscular system, the method comprising: obtaining, from a body, a neuromuscular system signal generated by one set of electrical stimulation (ES); generating comparison data between at least a portion of a signal of an early part and at least a portion of a signal of a later part of the neuromuscular system signal; generating a feature vector based on the generated comparison data; and evaluating the neuromuscular system by inputting the generated feature vector into an artificial intelligence model. Also, the one set of electrical stimulation (ES) may be electrical stimulation (ES) of the same intensity. Also, the method of evaluating the neuromuscular system may comprise obtaining a neuromuscular system signal for each of a plurality of sets of electrical stimulation (ES) generated by applying a plurality of sets of electrical stimulation (ES) to a muscle, generating comparison data between at least a portion of a signal of an early part and at least a portion of a signal of a later part of the neuromuscular system signal, generating a feature vector based on the generated comparison data, and evaluating the neuromuscular system by inputting the generated feature vector into an artificial intelligence model, wherein each of the plurality of sets of electrical stimulation (ES) may have a different frequency from one another. Also, stimulations within a predetermined number of times among a first set of electrical stimulation (ES) may be the early part, and stimulations after the predetermined number of times among the first set of electrical stimulation (ES) may be the later part. Also, the generating of comparison data between at least a portion of a signal of an early part and at least a portion of a signal of a later part of the neuromuscular system signal may generate comparison data between the neuromuscular system signal acquired for a first stimulation of a first set of electrical stimulation (ES) and the neuromuscular system signal acquired for a last stimulation of the first set of electrical stimulation (ES). Also, the generating of the comparison data may generate the comparison data based on an amplitude of the signal. Also, the generating of the comparison data may generate the comparison data based on zero-crossing data, which is frequency data regarding the number of times a signal crosses a horizontal line. Also, the generating of the comparison data may generate the comparison data based on mean absolute value data, which is an index representing an average of absolute values of a signal. Also, the generating of the comparison data may generate the comparison data based on slope sign change data, which is data regarding the number of times the sign of a slope of a signal changes. Also, the generating of the comparison data may generate the comparison data based on Willison amplitude data, which is data regarding the degree to which changes exceeding a given threshold value appear within a signal. Also, the generating of the comparison data may generate the comparison data based on variance data, which is data representing the variability of a data set. Also, each of the plurality of sets of electrical stimulation (ES) may be electrical stimulation (ES) applied for the same duration.

Another embodiment of the present disclosure for solving the foregoing problems may provide a device for evaluating a neuromuscular system, the device comprising: a processor configured to acquire, from a body, a neuromuscular system signal generated by one set of electrical stimulation (ES), generate comparison data between at least a portion of a signal of an early part and at least a portion of a signal of a later part of the neuromuscular system signal, generate a feature vector based on the generated comparison data, and evaluate the neuromuscular system by inputting the generated feature vector into an artificial intelligence model. Also, the one set of electrical stimulation (ES) may be electrical stimulation (ES) of the same frequency. Also, the device may acquire a neuromuscular system signal for each of a plurality of sets of electrical stimulation (ES) generated by applying a plurality of sets of electrical stimulation (ES) to a muscle, generate comparison data between at least a portion of a signal of an early part and at least a portion of a signal of a later part of the neuromuscular system signal, generate a feature vector based on the generated comparison data, and evaluate the neuromuscular system by inputting the generated feature vector into an artificial intelligence model, wherein each of the plurality of sets of electrical stimulation (ES) may have a different frequency from one another. Also, stimulations within a predetermined number of times among a first set of electrical stimulation (ES) may be the early part, and stimulations after the predetermined number of times among the first set of electrical stimulation (ES) may be the later part.

Another embodiment of the present disclosure for solving the foregoing problems may provide a computer program stored on a computer-readable storage medium, the computer program comprising instructions for causing one or more processors to perform neuromuscular system condition evaluation, the instructions comprising: obtaining, from a body, a neuromuscular system signal generated by one set of electrical stimulation (ES); generating comparison data between at least a portion of a signal of an early part and at least a portion of a signal of a later part of the neuromuscular system signal; generating a feature vector based on the generated comparison data; and evaluating the neuromuscular system by inputting the generated feature vector into an artificial intelligence model. Also, the one set of electrical stimulation (ES) may be electrical stimulation (ES) of the same frequency. Also, the evaluating of the neuromuscular system may comprise obtaining a neuromuscular system signal for each of a plurality of sets of electrical stimulation (ES) generated by applying a plurality of sets of electrical stimulation (ES) to a muscle, generating comparison data between at least a portion of a signal of an early part and at least a portion of a signal of a later part of the neuromuscular system signal, generating a feature vector based on the generated comparison data, and evaluating the neuromuscular system by inputting the generated feature vector into an artificial intelligence model, wherein each of the plurality of sets of electrical stimulation (ES) may have a different frequency from one another. Also, stimulations within a predetermined number of times among a first set of electrical stimulation (ES) may be the early part, and stimulations after the predetermined number of times among the first set of electrical stimulation (ES) may be the later part.

Hereinafter, embodiments for solving one of the technical problems of the present disclosure will be described in detail.

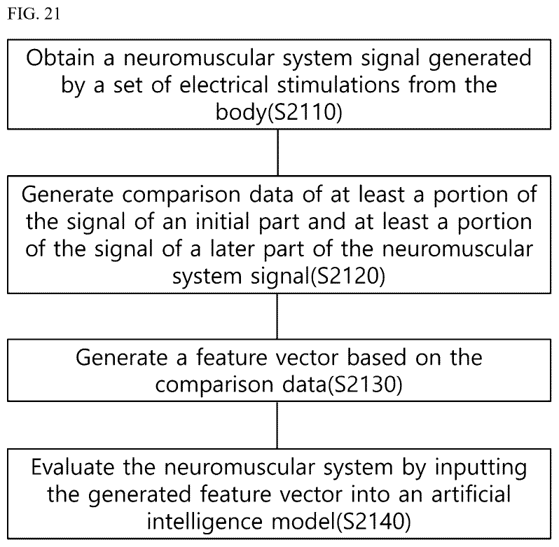

FIG. 21 is a diagram for explaining a method of evaluating a neuromuscular system according to an embodiment of the present disclosure.

According to an embodiment of the present disclosure, in step S2110, the device (100) may acquire, from a body, a neuromuscular system signal generated by one set of electrical stimulation (ES). For example, the device (100) may apply one set of electrical stimulation (ES) to the body and acquire an electrical signal that has passed through the human body as a neuromuscular system signal. In this case, the electrical stimulation and measurement unit (170) of the device (100) may apply electrical stimulation (ES) to a muscle.

According to another embodiment of the present disclosure, the device (100) may be physically separated from the electrical stimulation and measurement unit (170). For example, the device (100) may acquire a neuromuscular system signal from an external device. In this case, the device (100) may acquire the neuromuscular system signal through a wired and/or wireless network.

According to an embodiment of the present disclosure, one set of electrical stimulation (ES) may comprise a plurality of stimulations. For example, one set of electrical stimulation (ES) may consist of eight electrical stimulations applied once per second.

Also, the one set of electrical stimulation (ES) may be electrical stimulation (ES) of the same intensity. For example, when one set of electrical stimulation (ES) consists of eight stimulations, all eight electrical stimulations may be of the same intensity.

According to another embodiment of the present disclosure, the device (100) may apply a plurality of sets of electrical stimulation (ES). In this case, each of the plurality of sets of electrical stimulation (ES) may have a different frequency.

For example, the device (100) may sequentially apply to the body sets of electrical stimulation (ES) increasing by 5 Hz, such as one set of electrical stimulation (ES) at 5 Hz, one set at 10 Hz, one set at 15 Hz, one set at 20 Hz, and one set at 25 Hz. In this case, each set comprises a plurality of electrical stimulations, and the intensities of the plurality of electrical stimulations within each set may be the same.

In this case, the duration for which each of the plurality of sets applies electrical stimulation (ES) may be the same. For example, the duration for which one set of electrical stimulation (ES) at 5 Hz is applied and the duration for which one set of electrical stimulation (ES) at 10 Hz is applied may be the same.

In step S2120, the device (100) may generate comparison data between at least a portion of a signal of an early part and at least a portion of a signal of a later part of the neuromuscular system signal.

According to an embodiment of the present disclosure, in order to evaluate the neuromuscular system, the device (100) may utilize a portion of the acquired neuromuscular system signal. For example, the device (100) may generate comparison data between at least a portion of a signal of an early part and at least a portion of a signal of a later part of the neuromuscular system signal.

In this case, stimulations within a predetermined number of times among first set of electrical stimulation (ES) may be the early part, and stimulations after the predetermined number of times among first set of electrical stimulation (ES) may be the later part. Also, stimulations within a predetermined time among first set of electrical stimulation (ES) may be the early part, and stimulations after the predetermined time among first set of electrical stimulation (ES) may be the later part.

As a specific example, the device (100) may utilize the neuromuscular system signal acquired for a first stimulation of first set of electrical stimulation (ES) and the neuromuscular system signal acquired for a last stimulation of first set of electrical stimulation (ES).

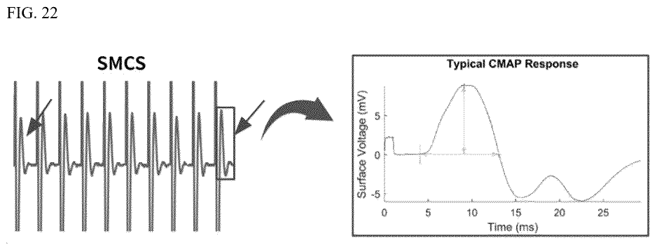

Referring to FIG. 22, the device (100) may generate comparison data by utilizing a CMAP (Compound Muscle Action Potential) signal (a) acquired by the first stimulation of one set of electrical stimulation (ES) and a CMAP (Compound Muscle Action Potential) signal (b) acquired by the last stimulation.

According to an embodiment of the present disclosure, the device (100) may generate comparison data by extracting a feature from at least a portion of a signal of an early part of the neuromuscular system signal, extracting a feature from at least a portion of a signal of a later part, and comparing the extracted features.

For example, the device (100) may generate comparison data by applying the formula: (feature (signal of early part)-feature (signal of later part))/feature (signal of early part)× 100. Specifically, referring to FIG. 22, the comparison data may be generated by (feature (signal (a))-feature (signal (b)))/feature (signal (a))× 100.

In this case, the device (100) may utilize data on various characteristics of the signal. The device (100) may generate comparison data based on an amplitude of the signal.

For example, according to an embodiment of the present disclosure, the device (100) may acquire signal magnitude data, which is data representing the amplitude of the signal. Specifically, the device (100) may generate comparison data between signal magnitude data from the CMAP (Compound Muscle Action Potential) signal of the early part and signal magnitude data from the CMAP (Compound Muscle Action Potential) signal of the later part of the neuromuscular system signal, and may generate a feature vector based on the generated comparison data. Also, the neuromuscular system may be evaluated by inputting the feature vector into an artificial intelligence model.

Also, the device (100) may generate comparison data based on zero-crossing data, which is frequency data regarding the number of times a signal crosses a horizontal line. A detailed description thereof will be provided below.

Also, the device (100) may generate comparison data based on mean absolute value data, which is data representing the average of absolute values of a signal. The mean absolute value is an index representing the average of absolute values of a signal and may indicate the activity level of the signal. Specifically, the device (100) may generate comparison data between mean absolute value data from the CMAP (Compound Muscle Action Potential) signal of the early part and mean absolute value data from the CMAP (Compound Muscle Action Potential) signal of the later part of the neuromuscular system signal, and may generate a feature vector based on the generated comparison data. Also, the neuromuscular system may be evaluated by inputting the feature vector into an artificial intelligence model.

Also, the device (100) may generate comparison data based on slope sign change data, which is data regarding the number of times the sign of a slope of a signal changes. A detailed description thereof will be provided below.

Also, the device (100) may generate comparison data based on Willison amplitude data, which is data regarding the degree to which changes exceeding a given threshold value appear within a signal. Willison amplitude data is data that measures how frequently changes exceeding a given threshold value occur within a signal, and indicates the activity level of the signal. For example, the more frequently changes exceeding a given threshold value occur, the higher the Willison amplitude may be. Specifically, the device (100) may generate comparison data between Willison amplitude data from the CMAP (Compound Muscle Action Potential) signal of the early part and Willison amplitude data from the CMAP (Compound Muscle Action Potential) signal of the later part of the neuromuscular system signal, and may generate a feature vector based on the generated comparison data. Also, the neuromuscular system may be evaluated by inputting the feature vector into an artificial intelligence model.

Also, the device (100) may generate comparison data based on variance data, which is data representing the variability of a data set. A detailed description thereof will be provided below.

In step S2130, the device (100) may generate a feature vector based on the comparison data. Also, in step S2140, the device (100) may evaluate the neuromuscular system by inputting the feature vector into an artificial intelligence model. An exemplary method of generating an artificial intelligence model has been described above.

According to an embodiment of the present disclosure, the device (100) may evaluate the neuromuscular system by utilizing a feature vector generated from at least one type of comparison data among various types of comparison data.

For example, the device (100) may evaluate the neuromuscular system by inputting into an artificial intelligence model at least one feature vector selected from: a feature vector obtained from comparison data generated based on an amplitude of the signal; a feature vector obtained from comparison data generated based on zero-crossing data; a feature vector obtained from comparison data generated based on mean absolute value data; a feature vector obtained from comparison data generated based on slope sign change data; a feature vector obtained from comparison data generated based on Willison amplitude data; and a feature vector obtained from comparison data generated based on variance data.

FIGS. 23A and 23B are diagrams showing a trend of neuromuscular system signals according to an embodiment of the present disclosure.

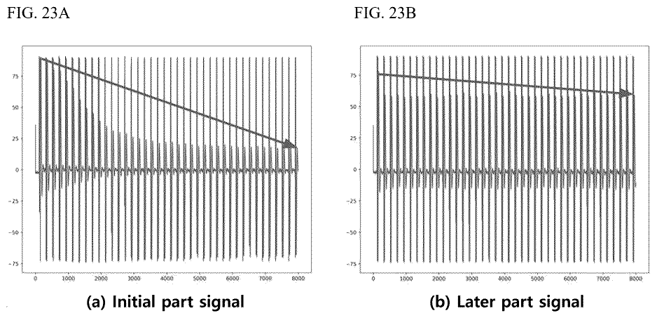

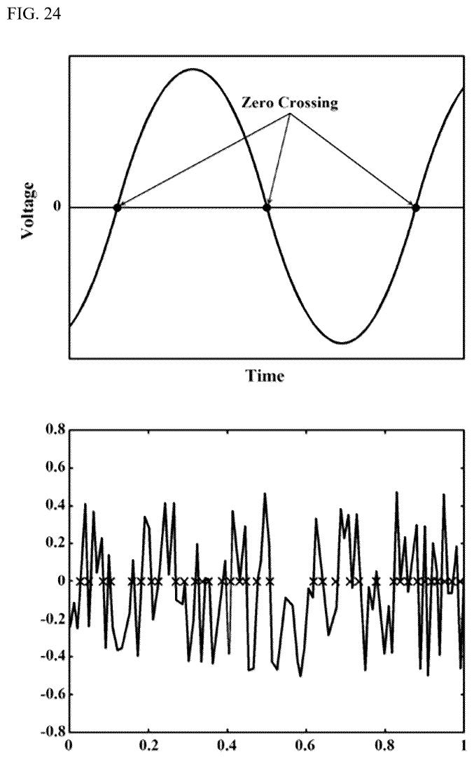

According to an embodiment of the present disclosure, FIG. 23A is a diagram showing a neuromuscular system signal in a neuromuscular degeneration state, and FIG. 23B is a diagram showing a neuromuscular system signal in a normal state. In the neuromuscular degeneration state, it can be confirmed that the magnitude of the CMAP (Compound Muscle Action Potential) signal in the neuromuscular system signal decreases. As such, since the CMAP (Compound Muscle Action Potential) signal in the normal state and the CMAP (Compound Muscle Action Potential) signal in the degeneration state differ from each other, the degree of neuromuscular degeneration can be evaluated through the degree of change in the CMAP (Compound Muscle Action Potential) signal using the method described above. FIG. 24 is a diagram for explaining zero-crossing data according to an embodiment of the present disclosure.

Zero-crossing data is data that measures the number of times a signal crosses a horizontal line to identify periodicity, and the frequency varies depending on how the signal rises and falls within time-series data. The portion marked X in FIG. 24 can be identified as the portion where zero-crossing occurs.

According to an embodiment of the present disclosure, the device (100) may acquire zero-crossing occurrence data, which is data regarding the degree to which zero-crossing occurs in the neuromuscular system signal. Specifically, the device (100) may generate comparison data between zero-crossing data from the CMAP (Compound Muscle Action Potential) signal of the early part and zero-crossing data from the CMAP (Compound Muscle Action Potential) signal of the later part of the neuromuscular system signal, and may generate a feature vector based on the generated comparison data. Also, the neuromuscular system may be evaluated by inputting the feature vector into an artificial intelligence model.

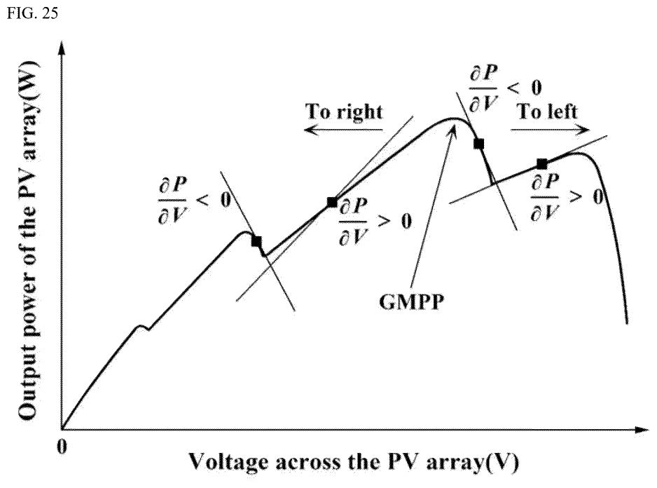

FIG. 25 is a diagram for explaining slope sign change data according to an embodiment of the present disclosure.

Slope sign change data is data that measures the number of times the sign of a slope of a signal changes to represent the complexity of the signal, and may indicate how rapidly the signal changes within time-series data. FIG. 25 exemplarily illustrates a method of measuring the slope at each part of the signal.

According to an embodiment of the present disclosure, the device (100) may acquire slope sign change data, which is data regarding the number of times the sign of a slope changes in the neuromuscular system signal. Specifically, the device (100) may generate comparison data between slope sign change data from the CMAP (Compound Muscle Action Potential) signal of the early part and slope sign change data from the CMAP (Compound Muscle Action Potential) signal of the later part of the neuromuscular system signal, and may generate a feature vector based on the generated comparison data. Also, the neuromuscular system may be evaluated by inputting the feature vector into an artificial intelligence model.

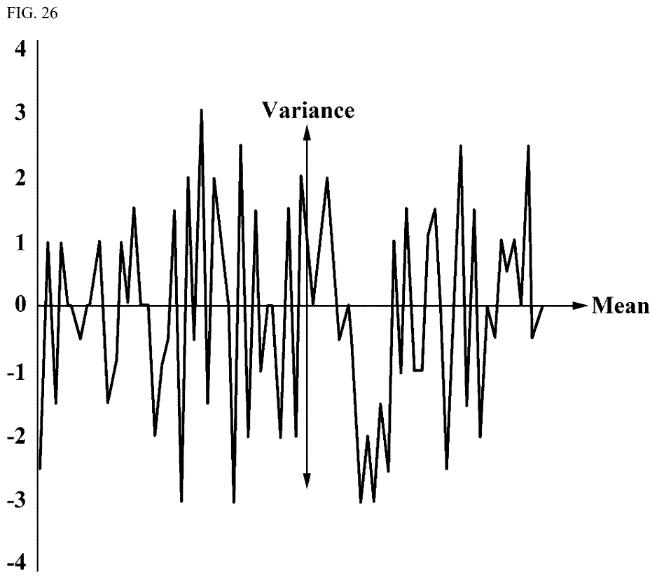

FIG. 26 is a diagram for explaining variance data according to an embodiment of the present disclosure.

Variance data is data representing the variability of a signal, and may indicate how spread out the signal is from its mean value. FIG. 26 exemplarily illustrates a method of measuring variance data.

According to an embodiment of the present disclosure, the device (100) may acquire variance data, which is data representing the variability of a data set. Specifically, the device (100) may generate comparison data between variance data from the CMAP (Compound Muscle Action Potential) signal of the early part and variance data from the CMAP (Compound Muscle Action Potential) signal of the later part of the neuromuscular system signal, and may generate a feature vector based on the generated comparison data. Also, the neuromuscular system may be evaluated by inputting the feature vector into an artificial intelligence model.

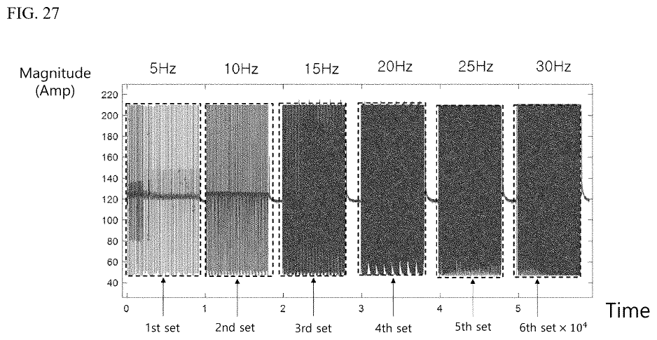

FIG. 27 is a diagram for explaining an embodiment in which a device applies a plurality of sets of electrical stimulation (ES) according to an embodiment of the present disclosure.

According to an embodiment of the present disclosure, the device (100) may apply a plurality of sets of electrical stimulation (ES). In this case, each of the plurality of sets of electrical stimulation (ES) may have a different frequency.

For example, the device (100) may sequentially apply to the body sets of electrical stimulation (ES) increasing by 5 Hz, such as one set of electrical stimulation (ES) at 5 Hz, one set at 10 Hz, one set at 15 Hz, one set at 20 Hz, and one set at 25 Hz. In this case, each set comprises a plurality of electrical stimulations, and the intensities of the plurality of electrical stimulations within each set may be the same.

In this case, the duration for which each of the plurality of sets applies electrical stimulation (ES) may be the same. For example, the duration for which one set of electrical stimulation (ES) at 5 Hz is applied and the duration for which one set of electrical stimulation (ES) at 10 Hz is applied may be the same.

The device (100) may acquire a neuromuscular system signal for each of a plurality of sets of electrical stimulation (ES) generated by applying the plurality of sets of electrical stimulation (ES) to a muscle.

The device (100) may generate comparison data between at least a portion of a signal of an early part and at least a portion of a signal of a later part of the neuromuscular system signal, generate a feature vector based on the generated comparison data, and evaluate the neuromuscular system by inputting the generated feature vector into an artificial intelligence model.

For example, the device (100) may select a neuromuscular system signal generated by at least one electrical stimulation among a first set, a second set, a third set, . . . , and an n-th set (n being a natural number). Also, the device (100) may generate comparison data between at least a portion of a signal of an early part and at least a portion of a signal of a later part of the selected neuromuscular system signal, generate a feature vector based on the generated comparison data, and evaluate the neuromuscular system by inputting the generated feature vector into an artificial intelligence model. In this case, the frequencies of the electrical stimulation of the first set, the second set, the third set, . . . , and the n-th set (n being a natural number) may be different from one another.

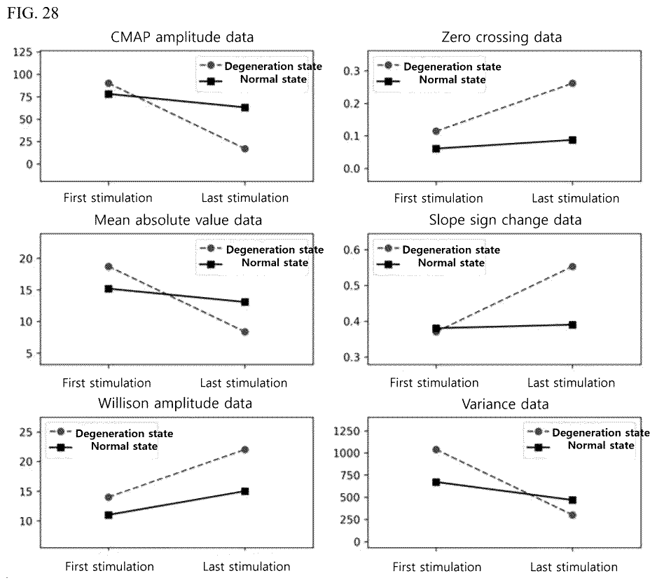

FIG. 28 is a diagram for comparing a neuromuscular system signal generated by a first stimulation and a neuromuscular system signal generated by a last stimulation according to an embodiment of the present disclosure.

According to an embodiment of the present disclosure, by comparing a feature extracted from a CMAP signal generated by a first stimulation of one set of electrical stimulation (ES) and a feature extracted from a CMAP signal generated by a last stimulation, a difference between a normal muscle state and a muscle degeneration state can be confirmed.

Accordingly, by utilizing the comparison data as a feature vector and using it as input data for an artificial intelligence model, the artificial intelligence model can predict whether a user's muscle has degenerated.

An embodiment for solving one of the technical problems of the present disclosure may provide a method and apparatus for neuromuscular system condition evaluation, such as muscle quality diagnosis, by analyzing a neuromuscular system signal based on electrical stimulation (ES).

An embodiment of the present disclosure for solving the foregoing problems may provide a method by which a device evaluates a neuromuscular system, the method comprising: a neuromuscular system signal acquisition step of obtaining a neuromuscular system signal with respect to electrical stimulation (ES); a step of generating feature information by preprocessing the acquired neuromuscular system signal through a data preprocessing method comprising a data dimensionality reduction algorithm that reduces the complexity of data; and a step of evaluating the neuromuscular system by inputting the feature information and personal information of a user into an artificial intelligence model. Also, the step of generating the feature information may comprise: a step of generating first feature information generated by preprocessing the acquired neuromuscular system signal through a first preprocessing method; and a step of generating second feature information generated by preprocessing the acquired neuromuscular system signal through a second preprocessing method; and the step of evaluating the neuromuscular system may evaluate the neuromuscular system by inputting the first feature information, the second feature information, and the personal information of the user into an artificial intelligence model, wherein the first preprocessing method and the second preprocessing method may be different from each other. Also, the neuromuscular system signal may be a neuromuscular system signal for multi-frequency. Also, the first preprocessing method may preprocess the neuromuscular system signal in a time domain or a frequency domain, and the second preprocessing method may preprocess the neuromuscular system signal by converting a time-domain neuromuscular system signal into two-dimensional data in a time-frequency domain. Also, the first preprocessing method may comprise a Cepstrum method. Also, the first preprocessing method may comprise a 1D-autoencoder method. Also, the second preprocessing method may convert the neuromuscular system signal into two-dimensional data in a time-frequency domain and extract feature information from the converted two-dimensional data. Also, the second preprocessing method may extract feature information using at least one of a 2D-autoencoder method and a pattern extraction method. Also, the personal information may comprise at least one of weight information of the user, age information of the user, height information of the user, gender information, medical history information of the user, and medical data of the user. Also, the Cepstrum method may comprise: obtaining a neuromuscular system signal; performing a Fourier Transform on the neuromuscular system signal to generate FT neuromuscular system signal information; calculating a log spectrum of the neuromuscular system signal by applying a logarithmic function to the FT neuromuscular system signal information; performing an Inverse Fourier Transform on the calculated log spectrum to obtain CA (cepstrum analysis) coefficient information; obtaining feature information based on the CA (cepstrum analysis) coefficient information; and evaluating the neuromuscular system based on the obtained feature information. Also, the step of obtaining the feature information may generate preprocessed CA coefficient information by removing an FF (fundamental frequency) component corresponding to a frequency of the electrical stimulation (ES) from the CA (cepstrum analysis) coefficient information and further removing an HF (harmonic frequency) component from the CA (cepstrum analysis) coefficient information, and may obtain feature information based on the preprocessed CA coefficient information.

Another embodiment of the present disclosure for solving the foregoing problems may provide a device for evaluating a neuromuscular system, the device comprising: a processor configured to acquire a neuromuscular system signal with respect to electrical stimulation (ES), generate feature information by preprocessing the acquired neuromuscular system signal through a data preprocessing method comprising a data dimensionality reduction algorithm that reduces the complexity of data, and evaluate the neuromuscular system by inputting the feature information and personal information of a user into an artificial intelligence model. Also, the processor may generate first feature information by preprocessing the acquired neuromuscular system signal through a first preprocessing method, generate second feature information by preprocessing the acquired neuromuscular system signal through a second preprocessing method, and evaluate the neuromuscular system by inputting the first feature information, the second feature information, and the personal information of the user into an artificial intelligence model, wherein the first preprocessing method and the second preprocessing method may be different from each other. Also, the first preprocessing method may preprocess the neuromuscular system signal in a time domain or a frequency domain, and the second preprocessing method may preprocess the neuromuscular system signal by converting one-dimensional data in a time domain into two-dimensional data in a time-frequency domain. Also, the first preprocessing method may comprise a Cepstrum method. Also, the first preprocessing method may comprise a 1D-autoencoder method. Also, the second preprocessing method may convert the neuromuscular system signal into two-dimensional data in a time-frequency domain and extract feature information from the converted two-dimensional data. Also, the second preprocessing method may extract feature information using at least one of a pattern extraction method and a 2D-autoencoder method. Also, the personal information may comprise at least one of weight information of the user, age information of the user, height information of the user, and medical history information of the user.

Another embodiment of the present disclosure for solving the foregoing problems may provide a computer program stored on a computer-readable storage medium, the computer program comprising instructions for causing one or more processors to perform neuromuscular system condition evaluation, the instructions comprising: a neuromuscular system signal acquisition step of obtaining a neuromuscular system signal with respect to electrical stimulation (ES); a step of generating feature information by preprocessing the acquired neuromuscular system signal through a data preprocessing method comprising a data dimensionality reduction algorithm that reduces the complexity of data; and a step of evaluating the neuromuscular system by inputting the feature information and personal information of a user into an artificial intelligence model. Also, the step of generating the feature information may comprise: a step of generating first feature information generated by preprocessing the acquired neuromuscular system signal through a first preprocessing method; and a step of generating second feature information generated by preprocessing the acquired neuromuscular system signal through a second preprocessing method; and the step of evaluating the neuromuscular system may evaluate the neuromuscular system by inputting the first feature information, the second feature information, and the personal information of the user into an artificial intelligence model, wherein the first preprocessing method and the second preprocessing method may be different from each other. Also, the first preprocessing method may preprocess the neuromuscular system signal in a time domain or a frequency domain, and the second preprocessing method may preprocess the neuromuscular system signal by converting one-dimensional data in a time domain into two-dimensional data in a time-frequency domain. Also, the first preprocessing method may comprise a Cepstrum method. Also, the first preprocessing method may comprise a 1D-autoencoder method.

Hereinafter, detailed descriptions for explaining the above-described embodiments will be provided.

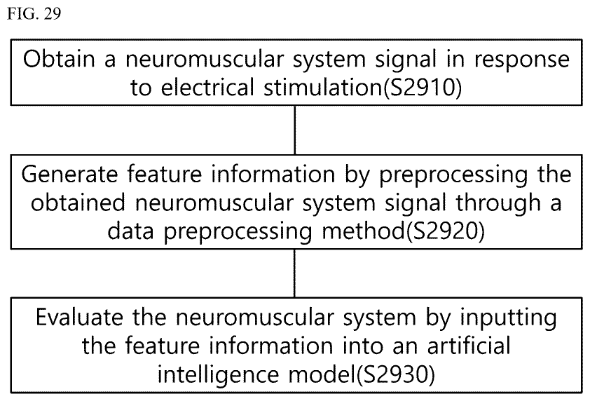

FIG. 29 is a diagram for explaining a method of evaluating a neuromuscular system according to an embodiment of the present disclosure.

According to an embodiment of the present disclosure, in step S2910, the device (100) may acquire a neuromuscular system signal with respect to electrical stimulation (ES).

The device (100) may acquire a neuromuscular system signal for a single frequency, and may acquire a neuromuscular system signal for multi-frequency.

For example, the device (100) may apply a single-frequency electrical stimulation (ES) to the body and acquire a neuromuscular system signal from the body as a response thereto.

As another example, the device (100) may apply a plurality of sets of electrical stimulation (ES) to the body. In this case, each of the plurality of sets of electrical stimulation (ES) may have a different frequency. Specifically, the device (100) may sequentially apply to the body sets of electrical stimulation (ES) increasing by 5 Hz, such as one set of electrical stimulation (ES) at 5 Hz, one set at 10 Hz, one set at 15 Hz, one set at 20 Hz, and one set at 25 Hz. Also, in response thereto, the device (100) may acquire a neuromuscular system signal from the body.

In step S2920, the device (100) may generate feature information by preprocessing the acquired neuromuscular system signal through a data preprocessing method.

The data preprocessing method may comprise a data dimensionality reduction algorithm that reduces the complexity of data.

For example, the data dimensionality reduction algorithm may comprise a Manifold Learning method.

The Manifold Learning method is a nonlinear dimensionality reduction technique for discovering and analyzing the intrinsic low-dimensional structure of high-dimensional data. Under the assumption that data often exists in a high-dimensional space but is actually formed in a lower-dimensional space, the core of Manifold Learning is to effectively learn this low-dimensional structure. This method reduces dimensionality while preserving the fundamental geometric properties of the data, and can be usefully applied to various tasks such as data visualization, noise removal, feature extraction, and data compression. Representative Manifold Learning algorithms include Isomap, Locally Linear Embedding (LLE), t-Distributed Stochastic Neighbor Embedding (t-SNE), and Autoencoder (AE).

These algorithms map high-dimensional data to a low-dimensional space while maintaining the local and global structure of the data, enabling clearer identification of the latent patterns and structure of the data. For example, t-SNE and UMAP demonstrate excellent performance in data visualization and can effectively represent the clustering and distribution of high-dimensional data in two-dimensional or three-dimensional space.

According to an embodiment of the present disclosure, a latent vector, which is a low-dimensional spatial representation of an autoencoder, may be used as a feature extracted by the Manifold Learning method. In this case, the autoencoder (AE) may comprise variants of the autoencoder, such as a Variational Autoencoder (VAE) and a Conditional VAE (CVAE).

An autoencoder is a type of artificial neural network that learns important features of data through a process of compressing input data into a low-dimensional space and then reconstructing it. An autoencoder consists of two parts: an Encoder and a Decoder. The encoder transforms input data from a high-dimensional space to a low-dimensional space, generating a representation in a low-dimensional space called a latent space, i.e., a latent vector. This latent vector contains the core information of the input data and represents the important features of the data in a compressed form. The decoder transforms this latent vector back into a high-dimensional space to generate output similar to the original input data. Through this process, the autoencoder can effectively learn important features of input data and be applied in various fields such as noise removal, dimensionality reduction, data compression, and generative modeling.

A Variational Autoencoder (VAE) is an extended form of the autoencoder, and is a probabilistic model capable of better understanding the latent structure of data and generating new data. While maintaining the basic structure of an encoder and decoder, the VAE is distinguished by the fact that it encodes input data as a distribution rather than a single point. The encoder maps input data to a normal distribution (Gaussian distribution) in the latent space to generate a mean vector and a variance vector. A latent vector sampled from this distribution allows the data to reflect various variations.

The core idea of the VAE is to transform input data into a probability distribution in the latent space, and to train the decoder to generate data using a latent vector sampled from this distribution. This makes the VAE very useful for generating new data points. During training, the VAE optimizes two loss functions (reconstruction loss and regularization loss) that minimize the difference between the input data and the generated data while inducing the distribution of the latent space to follow a normal distribution. Through this process, the VAE forms a latent space that well reflects the features of the input data.

The VAE is used in various application domains, including image generation, data generation, noise removal, and anomaly detection. For example, by training a VAE on facial image data, new facial images can be generated or existing facial images can be transformed. The VAE also enables a deeper understanding of the latent structure of data through meaningful manipulation of the latent space. Due to these characteristics, the VAE plays an important role in the field of generative modeling.

A Conditional Variational Autoencoder (CVAE) is an extended form of the Variational Autoencoder (VAE), focusing on conditionally learning and generating the latent structure of data. The CVAE maintains the basic principles of the VAE while providing additional conditional information as input to the encoder and decoder to enable control over the data generation process. This conditional information may comprise labels, classes, or other contextual information, enabling the generation of data satisfying specific conditions.

The CVAE consists of an encoder and a decoder, where the encoder combines input data with conditional information and maps it to a normal distribution (Gaussian distribution) in the latent space. A mean vector and a variance vector are generated here, and the latent vector sampled from this distribution contains the latent representation of the data reflecting the conditional information. The decoder combines this latent vector with the conditional information to reconstruct the original input data or generate new data.

The training process of the CVAE, similarly to the VAE, optimizes two loss functions (reconstruction loss and regularization loss) that minimize the difference between the input data and the generated data while inducing the distribution of the latent space to follow a normal distribution. In this process, the conditional information helps the model generate data matching specific conditions.

The CVAE is useful in various application domains. For example, in the field of image generation, images of a specific class can be generated, and in the field of text generation, text reflecting a specific topic or style can be generated. Also, in problems such as data augmentation and anomaly detection, more accurate and useful results can be obtained by utilizing conditional information. By introducing conditional control into the data generation process, the CVAE greatly expands the flexibility and scope of application of generative modeling.

According to an embodiment of the present disclosure, a first preprocessing method and a second preprocessing method to be described later may comprise the data dimensionality reduction algorithm that reduces the complexity of data described above.

According to an embodiment of the present disclosure, the device (100) may generate first feature information generated by preprocessing the acquired neuromuscular system signal through a first preprocessing method.

For example, the device (100) may generate first feature information by preprocessing the neuromuscular system signal in a time domain or a frequency domain.

For example, the device (100) may generate first feature information by normalizing the neuromuscular system signal.

As another example, the device (100) may generate first feature information by performing at least one of a Cepstrum method and a 1D-autoencoder method on the neuromuscular system signal.

As another specific example, the device (100) may generate first feature information by normalizing the acquired neuromuscular system signal and performing a Cepstrum method on the normalized signal.

Also, the device (100) may generate first feature information by normalizing the acquired neuromuscular system signal and applying a 1D-autoencoder method to the normalized signal.

An autoencoder is an artificial intelligence model that learns input data and extracts feature information. For example, a 1D-autoencoder is an artificial intelligence model trained on one-dimensional data and can extract feature information from one-dimensional data. A 2D-autoencoder is an artificial intelligence model trained on two-dimensional data (e.g., images, etc.) and can extract feature information from two-dimensional data. A 2D-autoencoder can convert original data into less data while maximally retaining the information of the original data, and can achieve the effect of dimensionality reduction.

The device (100) may generate second feature information by preprocessing the acquired neuromuscular system signal through a second preprocessing method.

For example, the device (100) may convert the acquired neuromuscular system signal into two-dimensional data in a time-frequency domain and extract feature information from the converted two-dimensional data.

Specifically, the device (100) may convert the neuromuscular system signal into two-dimensional data in a time-frequency domain and extract feature information by applying at least one of a 2D-autoencoder method and a pattern extraction method to the two-dimensional data.

In this case, the device (100) may utilize STFT, CWT, or Wavelet transform to convert the data into two-dimensional data, and is not limited thereto and may utilize various methods.

A Short-Time Fourier Transform (STFT) can convert one-dimensional data in a time domain into two-dimensional data in a time-frequency domain. For example, the STFT can convert a neuromuscular system signal by separating it with a specific window size, performing a Fourier Transform on each segment to convert it into frequency components, and then concatenating these in the time domain. When a Fourier Transform is performed on the entire data, only the main frequency components can be identified with no temporal information; however, using the STFT has the advantage of being able to identify the main frequency components over time.

A Continuous Wavelet Transform (CWT) is also a technique, like the STFT, that converts one-dimensional data in a time domain into two-dimensional data in a time-frequency domain. The difference between the CWT and the STFT is that the CWT uses a wavelet instead of a Fourier Transform to identify the main frequency components and can generate two-dimensional data in a time-frequency domain with higher resolution. Another difference is that while the STFT has fixed time and frequency units for the generated two-dimensional data, the CWT can set the frequency unit more finely in the low-frequency range (in which case the time unit is larger) and the frequency unit larger in the high-frequency range (in which case the time unit is smaller).

In step S2930, the device (100) may evaluate the neuromuscular system by inputting the obtained feature information into an artificial intelligence model.

For example, the device (100) may evaluate the neuromuscular system by inputting extracted feature information into an artificial intelligence model trained through artificial intelligence-based model training.

As another example, the device (100) may evaluate the neuromuscular system signal by inputting feature information and personal information of a user into an artificial intelligence model.

For example, the device (100) may evaluate the neuromuscular system signal by inputting into an artificial intelligence model first feature information generated by preprocessing the neuromuscular system signal through a first preprocessing method, second feature information generated by preprocessing through a second preprocessing method, and personal information. In this case, the personal information of the user may comprise at least one of weight information of the user, age information of the user, height information of the user, gender information, medical history information of the user, and medical data of the user.

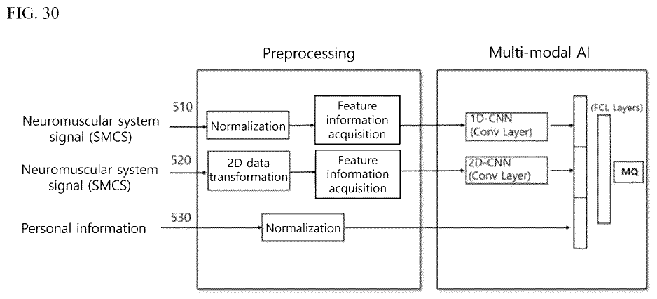

FIG. 30 is a diagram for explaining an artificial intelligence model according to an embodiment of the present disclosure.

According to an embodiment of the present disclosure, the device (100) may acquire a neuromuscular system signal with respect to multi-frequency electrical stimulation (ES).

Also, the device (100) may generate first feature information by preprocessing the acquired neuromuscular system signal through a first preprocessing method (510).

Also, the device (100) may generate second feature information by preprocessing the acquired neuromuscular system signal through a second preprocessing method. In this case, the first preprocessing method and the second preprocessing method may be different from each other (520).

The device (100) may evaluate the neuromuscular system by inputting the first feature information and the second feature information into an artificial intelligence model. In this case, the device (100) may evaluate the neuromuscular system by also inputting preprocessed personal information into the artificial intelligence model together (530).

Personal information refers to personal data about the user. For example, personal information may comprise at least one of weight information of the user, age information of the user, height information of the user, and medical history information of the user, and is not limited thereto and may comprise various personal data.

The device (100) may normalize the personal information and then input it into the artificial intelligence model.

In this case, the artificial intelligence model may comprise a first sub-model (e.g., a 1D-CNN (Convolutional Neural Network) in FIG. 30) that processes feature information generated by first preprocessing (e.g., preprocessed in a time domain or a frequency domain), and a second sub-model (e.g., a 2D-CNN (Convolutional Neural Network) in FIG. 30) that processes feature information generated by second preprocessing (e.g., preprocessed in a two-dimensional time-frequency domain).

Also, according to an embodiment of the present disclosure, the artificial intelligence model may evaluate a muscle condition by inputting result information of the first sub-model, result information of the second sub-model, and personal information into a classification model (e.g., FCL Layers in FIG. 30).

The Cepstrum method has been described with reference to FIGS. 15 through 19.

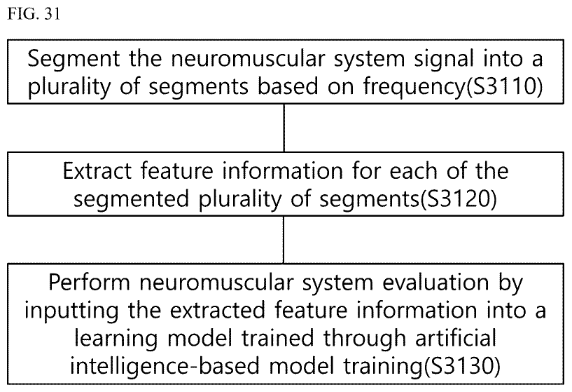

FIG. 31 is a diagram for explaining a pattern extraction method according to an embodiment of the present disclosure.

According to an embodiment of the present disclosure, in step S1010, the device (100) may divide the neuromuscular system signal into a plurality of segment units based on frequency.

According to another embodiment of the present disclosure, the device (100) may apply electrical stimulation (ES) of different frequencies to the user over time. For example, the device (100) may apply to the user electrical stimulation (ES) of a frequency that increases by a predetermined frequency over time. As a specific example, electrical stimulation (ES) of 5 Hz may be applied during a first time unit, electrical stimulation (ES) of 10 Hz during a second time unit, and electrical stimulation (ES) of 15 Hz during a third time unit.

In this case, the stimulated muscle contraction signal (SMCS) acquired by the device (100) may also comprise different frequencies over time. For example, a stimulated muscle contraction signal (SMCS) received in response to electrical stimulation (ES) of 5 Hz may comprise a frequency of 5 Hz, a stimulated muscle contraction signal (SMCS) received in response to electrical stimulation (ES) of 10 Hz may comprise a frequency of 10 Hz, and a stimulated muscle contraction signal (SMCS) received in response to electrical stimulation (ES) of 15 Hz may comprise a frequency of 15 Hz.

The device (100) may divide the acquired neuromuscular system signal into a plurality of segment units based on frequency. For example, the device (100) may divide the acquired neuromuscular system signal into a plurality of segments. In this case, signals included in each segment may have the same frequency. Also, in this case, signals included in each segment may be signals having frequencies within a specific range.

In step S1020, the device (100) may extract feature information for each of the divided plurality of segments.

The device (100) may convert the neuromuscular system signal included in each of the plurality of segments into a time-frequency domain. According to an embodiment of the present disclosure, the neuromuscular system signal acquired by the device (100) is a signal in a time-magnitude domain, and the acquired neuromuscular system signal may be converted into the time-frequency domain for analysis. In this case, the device (100) may perform a spectrogram transform, a Short-Time Fourier Transform (STFT), and/or a Continuous Wavelet Transform (CWT) to convert the signal into the time-frequency domain, but is not limited thereto and may convert the acquired neuromuscular system signal into the time-frequency domain using various methods.

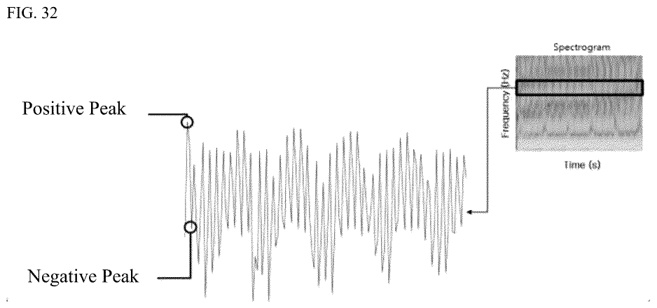

Referring to FIG. 32, the device (100) may obtain a positive peak and a negative peak from the neuromuscular system signal converted into the time-frequency domain. A positive peak refers to the peak at an inflection point where the signal rises and then falls, and a negative peak refers to the peak at an inflection point where the signal falls and then rises.

The device (100) may obtain an envelope peak based on at least one of the positive peak and the negative peak.

For example, the device (100) may obtain the positive peak as an envelope peak. Also, the device may obtain the negative peak as an envelope peak.

As another example, the device (100) may obtain an envelope peak by adding the magnitude values of adjacent positive peaks and negative peaks.

Also, as another example, the device (100) may obtain an envelope peak by calculating the average of the magnitude values of adjacent (e.g., neighboring) positive peaks and negative peaks. As another example, the device (100) may obtain an envelope peak by calculating the average of the magnitude values of a plurality of adjacent positive peaks and a plurality of adjacent negative peaks.

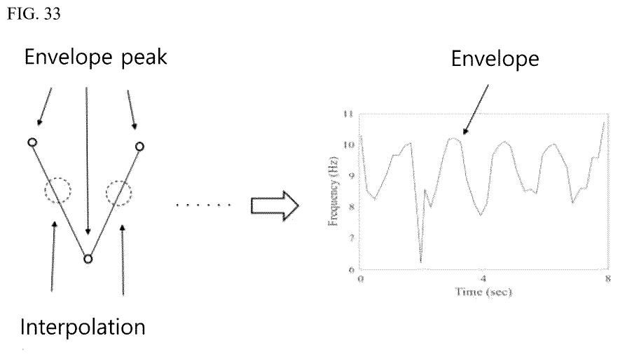

Referring to FIG. 33, the device (100) may obtain an envelope by interpolating the envelope peaks. In this case, the device (100) may apply at least one of Linear Interpolation, Parabolic Interpolation, Newton Interpolation, Lagrange Interpolation, and Spline Interpolation, and is not limited thereto and may interpolate the envelope peaks using various interpolation methods.

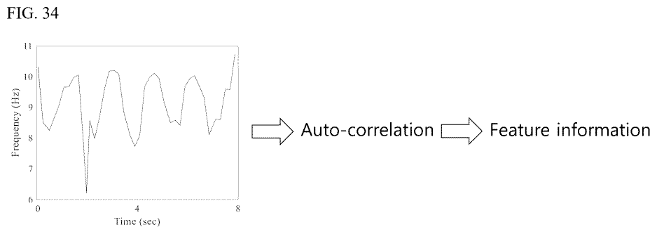

Referring to FIG. 34, the device (100) may extract feature information based on the generated envelope. In this case, the device (100) may obtain feature information by analyzing the pattern of the generated envelope.

In this case, the device (100) may extract feature information by performing at least one of sample entropy analysis, permutation entropy analysis, standard deviation signal analysis, square root analysis, auto-correlation analysis, cardinality analysis, and Poincaré plot analysis.

Individual differences in exercise posture, body structure, and physical ability are also factors that make quantitative evaluation of muscle fatigue difficult. Since the distribution of muscles and the way muscles are used differ from person to person, responses to muscle fatigue may differ even when performing the same exercise. These individual differences make it difficult to apply consistent criteria in the process of evaluating muscle fatigue. To overcome these difficulties, methods for quantitatively evaluating muscle fatigue by performing neuromuscular system condition evaluation using electrical stimulation (ES) have been continuously studied.

As an embodiment for solving one of the technical problems of the present disclosure, the present disclosure may provide a method and apparatus for evaluating a neuromuscular system by analyzing a response signal based on electrical stimulation (ES).

An embodiment of the present disclosure for solving the foregoing problems may provide a method by which a device evaluates a neuromuscular system, the method comprising: a muscle electrical stimulation step of applying muscle electrical stimulation (ES) to a muscle to induce muscle fatigue; a step of obtaining a neuromuscular system signal with respect to evaluation electrical stimulation (ES) after the muscle electrical stimulation step; and a step of evaluating the neuromuscular system based on the acquired neuromuscular system signal. Also, the step of evaluating the neuromuscular system may comprise: a step of obtaining intermediate evaluation data by calculating CA (cepstrum analysis) coefficient information based on the neuromuscular system signal; and evaluating the neuromuscular system based on the intermediate evaluation data. Also, the method by which the device evaluates the neuromuscular system may evaluate the neuromuscular system by applying a cumulative function to the intermediate evaluation data. Also, the method by which the device evaluates the neuromuscular system may evaluate the neuromuscular system by performing normalization on the data to which the cumulative function has been applied, obtaining a value of a median frequency, and obtaining a sum of the cumulative function up to the median frequency. Also, the muscle electrical stimulation (ES) for inducing muscle fatigue may have a longer stimulation duration than the evaluation electrical stimulation (ES). Also, the muscle electrical stimulation (ES) for inducing muscle fatigue may have a greater stimulation intensity than the evaluation electrical stimulation (ES). Also, the method by which the device evaluates the neuromuscular system may repeat the muscle electrical stimulation step and the signal acquisition step a plurality of times, and the step of evaluating the neuromuscular system may evaluate the neuromuscular system by utilizing at least one signal acquired through the plurality of repetitions. Also, the method may further comprise a pre-neuromuscular system signal acquisition step of obtaining a neuromuscular system signal with respect to evaluation electrical stimulation (ES) before the muscle electrical stimulation step, and the step of evaluating the neuromuscular system may evaluate the neuromuscular system based on the neuromuscular system signal with respect to the evaluation electrical stimulation (ES) acquired after the muscle electrical stimulation step and the neuromuscular system signal acquired in the pre-neuromuscular system signal acquisition step. Also, the step of obtaining intermediate evaluation data based on the neuromuscular system signal may comprise: performing a Fourier Transform on the neuromuscular system signal to generate FT neuromuscular system signal information; calculating a log spectrum of the neuromuscular system signal by applying a logarithmic function to the FT neuromuscular system signal information; performing an Inverse Fourier Transform on the calculated log spectrum to obtain CA (cepstrum analysis) coefficient information; and obtaining intermediate evaluation data based on the CA (cepstrum analysis) coefficient information. Also, the step of obtaining the intermediate information data may generate preprocessed CA coefficient information by removing an FF (fundamental frequency) component corresponding to the frequency of the electrical stimulation (ES) from the CA (cepstrum analysis) coefficient information, and may obtain intermediate evaluation data based on the preprocessed CA coefficient information. Also, the step of obtaining intermediate evaluation data may generate preprocessed CA coefficient information by further removing an HF (harmonic frequency) component generated by the electrical stimulation (ES) from the CA (cepstrum analysis) coefficient information, and may obtain intermediate evaluation data based on the preprocessed CA coefficient information. An embodiment of the present disclosure for solving the foregoing problems may provide a device for evaluating a neuromuscular system, the device comprising: a processor configured to evaluate the neuromuscular system based on a neuromuscular system signal acquired through a muscle electrical stimulation step of applying muscle electrical stimulation (ES) to a muscle to induce muscle fatigue and a step of obtaining a neuromuscular system signal with respect to evaluation electrical stimulation (ES) after the muscle stimulation step. Also, the processor may obtain intermediate evaluation data by calculating CA (cepstrum analysis) coefficient information based on the neuromuscular system signal, and may evaluate the neuromuscular system based on the intermediate evaluation data. Also, the processor may evaluate the neuromuscular system by applying a cumulative function to the intermediate evaluation data. Also, the device may evaluate the neuromuscular system by performing normalization on the data to which the cumulative function has been applied and obtaining a value of a median frequency.

Another embodiment of the present disclosure for solving the foregoing problems may provide a computer program stored on a computer-readable storage medium, the computer program comprising instructions for causing one or more processors to perform neuromuscular system condition evaluation, the instructions comprising: a muscle electrical stimulation step of applying muscle electrical stimulation (ES) to a muscle to induce muscle fatigue; and a step of obtaining a neuromuscular system signal with respect to evaluation electrical stimulation (ES) after the muscle stimulation step; and evaluating the neuromuscular system based on the neuromuscular system signal acquired through the foregoing steps. Also, the step of evaluating the neuromuscular system may obtain intermediate evaluation data by calculating CA (cepstrum analysis) coefficient information based on the neuromuscular system signal, and may evaluate the neuromuscular system by applying a cumulative function to the intermediate evaluation data. Also, the step of evaluating the neuromuscular system may evaluate the neuromuscular system by performing normalization on the data to which the cumulative function has been applied and obtaining a value of a median frequency.

Hereinafter, the above-described embodiments will be described in detail.

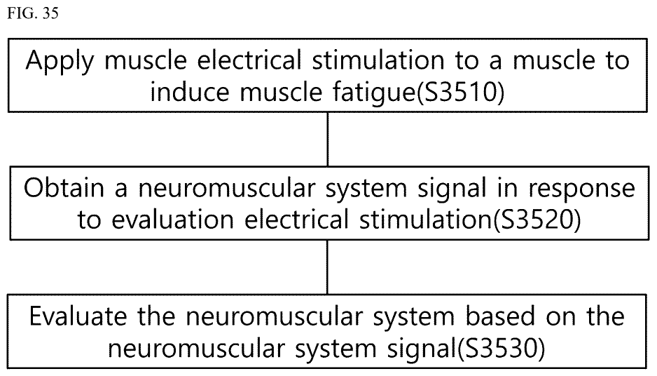

FIG. 35 is a diagram for explaining a method of evaluating a neuromuscular system according to an embodiment of the present disclosure.

According to an embodiment of the present disclosure, in the muscle electrical stimulation step of step S3510, the device (100) may apply muscle electrical stimulation (ES) to a muscle to induce muscle fatigue. For example, the electrical stimulation and measurement unit (170) of the device (100) may apply muscle electrical stimulation (ES) to a muscle to induce muscle fatigue.

According to another embodiment of the present disclosure, the device (100) may be physically separated from the electrical stimulation and measurement unit (170), and the electrical stimulation and measurement unit (170) may apply muscle electrical stimulation (ES) to a muscle to induce muscle fatigue.

In step S3520, the device (100) may acquire a neuromuscular system signal with respect to evaluation electrical stimulation (ES). In this case, the neuromuscular system signal may comprise the stimulated muscle contraction signal (SMCS) described above with reference to FIGS. 1A and 1B. Also, the evaluation electrical stimulation (ES) may be a single-frequency signal.

The device (100) may apply evaluation electrical stimulation (ES) to the user, and the stimulated muscle contraction signal (SMCS) acquired in response thereto may also comprise multiple frequencies.

In this case, the device (100) may apply evaluation electrical stimulation (ES) to the user a plurality of times, and may acquire the neuromuscular system signal a plurality of times in response thereto.

In step S3530, the device (100) may evaluate the neuromuscular system based on the acquired neuromuscular system signal.

The device (100) may obtain intermediate evaluation data based on the neuromuscular system signal, and may evaluate the neuromuscular system based on the intermediate evaluation data. The method of obtaining intermediate evaluation data will be described in detail with reference to FIG. 37.

FIG. 37 is a diagram for explaining a method for obtaining intermediate evaluation data according to an embodiment of the present disclosure.

In step S510, the device (100) may acquire a neuromuscular system signal. In this case, the neuromuscular system signal may comprise the stimulated muscle contraction signal (SMCS) described above with reference to FIGS. 1A and 1B. Also, the neuromuscular system signal may be a signal acquired from the body by applying a single-frequency electrical stimulation (ES).

The device (100) may apply multi-frequency electrical stimulation (ES) to the user. Also, a signal may be acquired from the neuromuscular system in response thereto. The acquired stimulated muscle contraction signal (SMCS) may comprise multiple frequencies. Also, the device (100) may apply single-frequency electrical stimulation (ES) and acquire a neuromuscular system signal from the body in response thereto.

Hereinafter, for convenience of explanation, a neuromuscular system signal acquired in response to multi-frequency electrical stimulation (ES) will be described. According to another embodiment of the present disclosure, the method of obtaining intermediate evaluation data described below may also be applied to a neuromuscular system signal acquired in response to single-frequency electrical stimulation (ES).

In step S520, the device (100) may obtain a log spectrum of the neuromuscular system signal and perform an Inverse Fourier Transform on the log spectrum to obtain CA (cepstrum analysis) coefficients.

According to an embodiment of the present disclosure, the device (100) may perform a Fourier Transform on the neuromuscular system signal to generate FT (Fourier Transform) neuromuscular system signal information (see FIG. 6).

According to an embodiment of the present disclosure, the device (100) may calculate a log spectrum of the neuromuscular system signal by applying a logarithmic function to the FT neuromuscular system signal information.

Also, the device (100) may perform an Inverse Fourier Transform on the calculated log spectrum to obtain CA (cepstrum analysis) coefficient information.

In this case, the device (100) may obtain CA coefficient information by utilizing complex values. Also, the device (100) may obtain CA coefficient information by utilizing real values. Also, the device (100) may obtain CA coefficient information by squaring the complex value and the real value respectively, adding them together, and utilizing the value obtained through a root operation.

Specifically, the device (100) may obtain CA coefficient information by extracting only the complex value from the result of performing an Inverse Fourier Transform on the calculated log spectrum. Also, the device (100) may obtain CA coefficient information by extracting only the real value from the result of performing an Inverse Fourier Transform on the calculated log spectrum. Also, the device (100) may obtain CA coefficient information by extracting a complex value and a real value from the result of performing an Inverse Fourier Transform on the calculated log spectrum, squaring the extracted complex value and the extracted real value respectively, adding them together, and performing a root operation (V {(complex value)× (complex value)+ (real value)× (real value)}).

The obtained CA coefficient information may be divided into an FF (fundamental frequency) component, an HF (harmonic frequency) component, and other components.

In step S530, the device (100) may obtain intermediate evaluation data based on the CA (cepstrum analysis) coefficient information.

According to an embodiment of the present disclosure, the device (100) may obtain the acquired CA (cepstrum analysis) coefficient information as intermediate evaluation data.

Also, the device (100) may perform preprocessing on the acquired CA (cepstrum analysis) coefficient information and obtain intermediate evaluation data based on the preprocessed CA (cepstrum analysis) coefficient information.

The device (100) may perform preprocessing on the acquired CA (cepstrum analysis) coefficient information.

For example, the device (100) may generate preprocessed CA coefficient information by removing an FF (fundamental frequency) component generated by the electrical stimulation (ES). Also, the device (100) may generate preprocessed CA coefficient information by removing an HF (harmonic frequency) component generated by the electrical stimulation (ES) from the CA coefficient information. Also, the device (100) may generate preprocessed CA coefficient information by removing both the FF (fundamental frequency) component and the HF (harmonic frequency) component generated by the electrical stimulation (ES) from the CA coefficient information. Also, the device (100) may obtain the preprocessed CA coefficient information as intermediate evaluation data.

According to an embodiment of the present disclosure, the FF (fundamental frequency) component may comprise information on the periodicity of the signal. For example, when there is a sine waveform oscillating 5 times per second, the FF (fundamental frequency) component appears at 5 Hz.

According to an embodiment of the present disclosure, the HF (harmonic frequency) component may comprise nonlinear characteristics. For example, when the FF (fundamental frequency) component has 5 Hz, the HF (harmonic frequency) component may occur at 10 Hz, 15 Hz, 20 Hz, . . . , 5xn Hz, and so on. As another example, when the FF (fundamental frequency) component is 10 Hz, the HF (harmonic frequency) component may occur at 20 Hz, 30 Hz, 40 Hz, . . . , 10xn Hz, and so on.

According to an embodiment of the present disclosure, the device (100) may obtain intermediate evaluation data based on the acquired CA coefficient information. Also, the device (100) may obtain intermediate evaluation data based on the preprocessed CA coefficient information.

In this case, the device (100) may obtain temporal change statistics information of the CA coefficient information (acquired CA coefficient information and/or preprocessed CA coefficient information), and obtain intermediate evaluation data based on the obtained temporal change statistics information. Specifically, the device (100) may obtain intermediate evaluation data based on at least one of a maximum value, a maximum value, a minimum value, a mean value, a median value, a standard deviation value, a variance value, an IQR (inter-quartile range) value, and a variance value of the CA coefficient information (acquired CA coefficient information and/or preprocessed CA coefficient information) over time.

Specifically, the device (100) may acquire a stimulated muscle contraction signal (SMCS) over a predetermined time interval (e.g., 8 seconds), and divide the acquired stimulated muscle contraction signal into a plurality of predetermined sub-time intervals (e.g., 1 second). Also, the device (100) may obtain candidate intermediate evaluation data for each sub-time interval by calculating CA coefficient information for each sub-time interval based on the method described above. Also, the device (100) may obtain temporal change statistics information of the candidate intermediate evaluation data obtained in each sub-time interval, and obtain intermediate evaluation data based on the obtained temporal change statistics information.

Also, the device (100) is not limited thereto and may obtain intermediate evaluation data using various methods.

In this case, the device (100) may apply multi-frequency electrical stimulation (ES) to the user a plurality of times, acquire the neuromuscular system signal a plurality of times in response thereto, and obtain intermediate evaluation data for each of the plurality of acquired neuromuscular system signals.

The method of obtaining CA (cepstrum analysis) coefficient information has been described above.

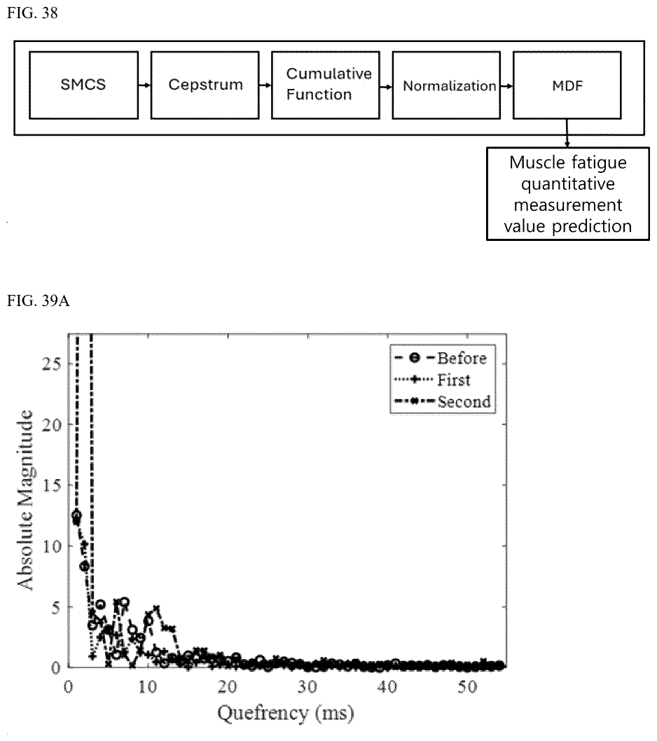

According to an embodiment of the present disclosure, referring to FIG. 38, the device (100) may apply a cumulative function to the obtained intermediate evaluation data. Also, the neuromuscular system may be evaluated by performing normalization on the data to which the cumulative function has been applied and obtaining a value of a median frequency.

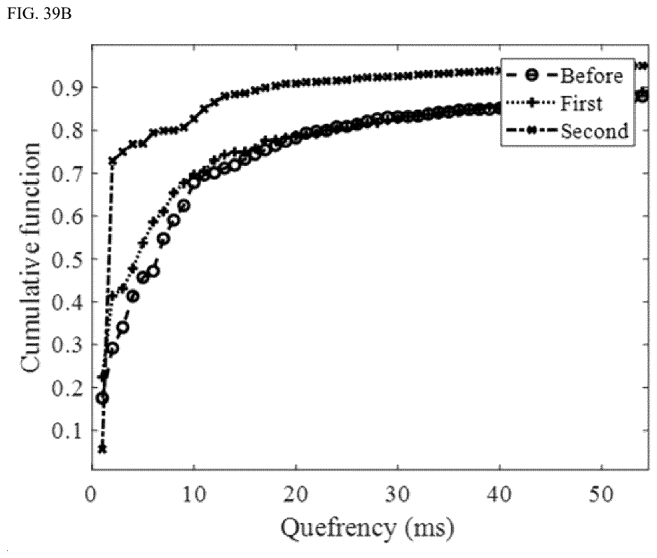

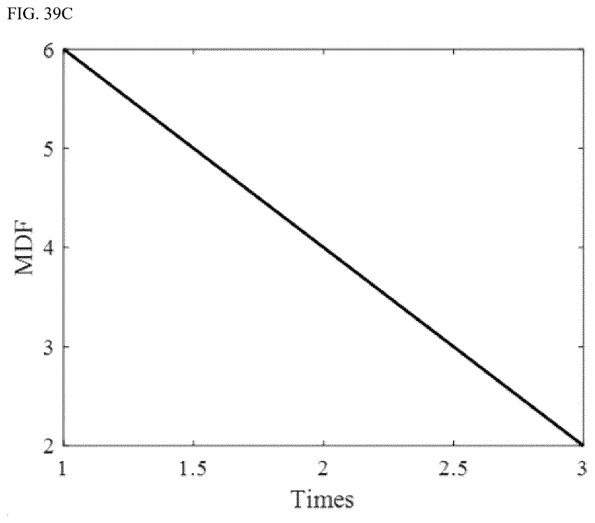

FIG. 39A shows intermediate evaluation data according to an embodiment of the present disclosure. For example, the device (100) may obtain intermediate evaluation data by removing an FF (fundamental frequency) component and an HF (harmonic frequency) component from the acquired CA coefficients.

FIG. 39B shows an embodiment in which a cumulative function is applied to intermediate evaluation data according to an embodiment of the present disclosure. In this case, the cumulative function may be expressed as

$$f ( k ) = {\sum }_{i = 0}^{k} ( x ( i ) ) .$$

In this case, k denotes an index of the cumulative function, and i in x (i) may denote a frequency bin.

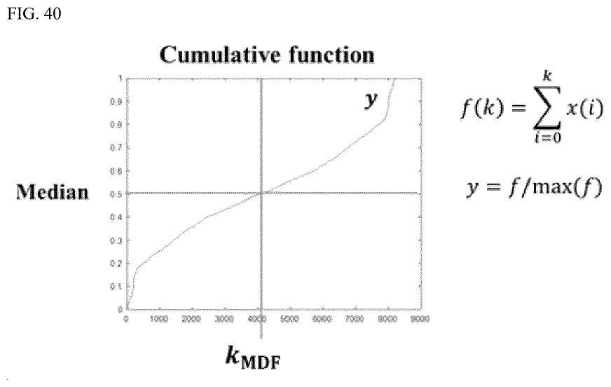

FIG. 40 is a diagram for explaining normalization being performed on data to which a cumulative function has been applied according to an embodiment of the present disclosure.

According to an embodiment of the present disclosure, normalized data y may be expressed as y=f/max (f). According to an embodiment of the present disclosure, the device (100) may obtain a frequency on the X-axis (e.g., k_{MDF} in FIG. 40) corresponding to a median value (e.g., a value corresponding to 0.5) of the normalized data y, and may determine a muscle fatigue quantitative measurement value based on the value of the obtained frequency. As a specific example, the muscle fatigue quantitative measurement value may be determined by the following formula.

$$Muscle fatigue quantative measurement value = {\sum }_{i = 0}^{{k}_{MDF}} y ( i )$$

In other words, the device (100) may determine a muscle fatigue quantitative value by obtaining the acquired k_{MDF} and obtaining the sum of the cumulative function up to k_{MDF}.

Referring to FIG. 39C, when the device (100) obtains a value of 6 in the first trial, a value of 4 in the second trial, and a value of 2 in the third trial, the device (100) may determine a muscle fatigue quantitative measurement difference value of 4 (value of first trial-value of third trial). In this case, the device (100) may evaluate the result more negatively as the difference value is larger.

According to another embodiment of the present disclosure, the device (100) may determine a muscle fatigue quantitative measurement value based on a difference in the acquired k_{MDF}. Specifically, the device (100) may determine a muscle fatigue quantitative measurement value based on a difference between k_{MDF} value of the first trial and k_{MDF} value of the last trial.

In this case, the device (100) may quantitatively evaluate muscle fatigue based on the muscle fatigue quantitative measurement value.

According to another embodiment of the present disclosure, the muscle electrical stimulation (ES) for inducing muscle fatigue may have a greater stimulation intensity than the evaluation electrical stimulation (ES). For example, the current of the electrical stimulation (ES) applied in the muscle electrical stimulation may be greater than the current of the evaluation electrical stimulation (ES). Also, the voltage of the electrical stimulation (ES) applied in the muscle electrical stimulation may be higher than the voltage of the evaluation electrical stimulation (ES).

According to an embodiment of the present disclosure, the muscle electrical stimulation (ES) for inducing muscle fatigue may have a longer stimulation duration than the evaluation electrical stimulation (ES). For example, if the total duration of the muscle electrical stimulation (ES) is 20 seconds, the total duration of the evaluation electrical stimulation (ES) may be 10 seconds.

In this case, the muscle electrical stimulation (ES) may be performed a plurality of times, and the evaluation electrical stimulation (ES) may also be performed a plurality of times. For example, the number of times of muscle electrical stimulation (ES) may be performed a plurality of times, and the evaluation electrical stimulation (ES) may also be performed a plurality of times; however, the total stimulation duration of the muscle electrical stimulation (ES) may be longer than the total duration of the evaluation electrical stimulation (ES).

According to an embodiment of the present disclosure, the device (100) may repeat the muscle electrical stimulation step and the signal acquisition step a plurality of times, and the step of evaluating the neuromuscular system may evaluate the neuromuscular system by utilizing all signals acquired through the plurality of repetitions. Also, according to an embodiment of the present disclosure, the method by which the device (100) evaluates the neuromuscular system may further comprise a pre-neuromuscular system signal acquisition step of obtaining a neuromuscular system signal with respect to evaluation electrical stimulation (ES) before the muscle electrical stimulation step. Also, the step of evaluating the neuromuscular system may evaluate the neuromuscular system based on the neuromuscular system signal with respect to the evaluation electrical stimulation (ES) acquired after the muscle electrical stimulation step and the neuromuscular system signal acquired in the pre-neuromuscular system signal acquisition step. A detailed description thereof will be provided below.

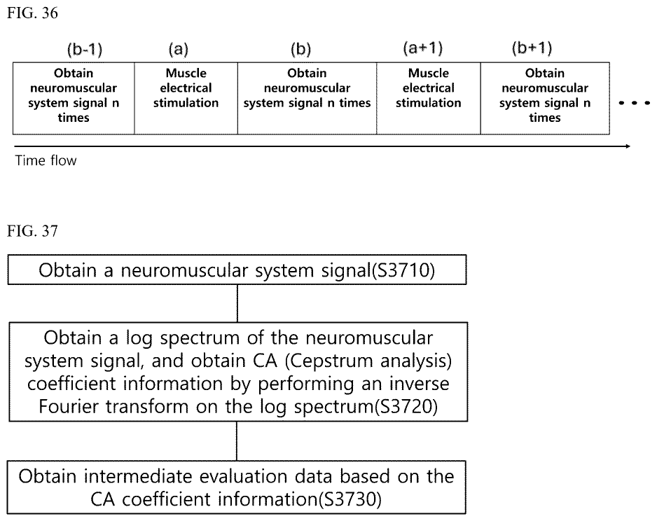

FIG. 36 is a diagram for explaining acquisition of a neuromuscular system signal using muscle electrical stimulation (ES) and evaluation electrical stimulation (ES).

Referring to FIG. 36, the device (100) may acquire a neuromuscular system signal before applying muscle electrical stimulation (ES). In this case, the device (100) may apply evaluation electrical stimulation (ES) to the user and acquire a neuromuscular system signal (b−1) from the user in response thereto. The device (100) may apply evaluation electrical stimulation (ES) n times (n being a natural number of 1 or more) and may acquire the neuromuscular system signal n times. In this case, the step of obtaining a neuromuscular system signal before applying the muscle electrical stimulation signal may be referred to as the pre-neuromuscular system signal acquisition step.

The device (100) may apply muscle electrical stimulation (ES) (a) to the user. In this case, the muscle electrical stimulation (ES) may be applied a plurality of times. In this case, the intensity of the muscle electrical stimulation (ES) may be greater than that of the evaluation electrical stimulation (ES). Also, the duration of application of the muscle electrical stimulation (ES) may be longer than the duration of application of the evaluation electrical stimulation (ES).

The device (100) may acquire a neuromuscular system signal after applying the muscle electrical stimulation (ES) (b). In this case, the device (100) may apply evaluation electrical stimulation (ES) to the user and acquire a neuromuscular system signal (b) from the user in response thereto. The device (100) may apply evaluation electrical stimulation (ES) n times (n being a natural number of 1 or more) and may acquire the neuromuscular system signal n times.

According to an embodiment of the present disclosure, the device (100) may repeat the step of applying muscle electrical stimulation (ES) and the step of obtaining a neuromuscular system signal a plurality of times. Although FIG. 36 only illustrates up to steps a+1 and b+1 due to space constraints, the device (100) may continue to repeat the step of applying muscle electrical stimulation (ES) and the step of obtaining a neuromuscular system signal a plurality of times up to a+n and b+n (n being a natural number of 1 or more).

The device (100) may perform neuromuscular system condition evaluation by utilizing at least a portion of the acquired neuromuscular system signals.

For example, the device (100) may perform neuromuscular system condition evaluation based on the neuromuscular system signal acquired in step (b). Also, the device (100) may perform neuromuscular system condition evaluation based on the neuromuscular system signals acquired in steps (b−1) and (b). Also, the device (100) may perform neuromuscular system condition evaluation based on the neuromuscular system signals acquired in steps (b−1) and (b+n) (in this case, b+n denotes the last instance of obtaining a neuromuscular system signal).

Also, the device (100) may perform neuromuscular system condition evaluation based on all neuromuscular system signals acquired in steps (b−1), (b), (b+1), . . . , and (b+n).

Also, the device (100) may select only signals whose intensity is equal to or greater than a predetermined intensity from among the neuromuscular system signals acquired in steps (b−1), (b), (b+1), . . . , and (b+n), and may perform neuromuscular system condition evaluation based on the selected neuromuscular system signals. The method of performing neuromuscular system condition evaluation based on the acquired neuromuscular system signal has been described above.

For convenience of explanation, the present disclosure has described muscle electrical stimulation (ES) application, neuromuscular system signal acquisition, and neuromuscular system condition evaluation as all being performed by the device (100); however, according to another embodiment of the present disclosure, the device that applies muscle electrical stimulation (ES) and acquires neuromuscular system signals and the device that performs neuromuscular system condition evaluation may be physically separate. Also, according to another embodiment of the present disclosure, the device that applies muscle electrical stimulation (ES) and the device that acquires neuromuscular system signals may also be physically separate.

According to an embodiment of the present disclosure, the foregoing methods may be performed by the processor (110) of the device (100). A detailed description of the physical configuration of the processor (110) has been provided above with reference to FIG. 2.

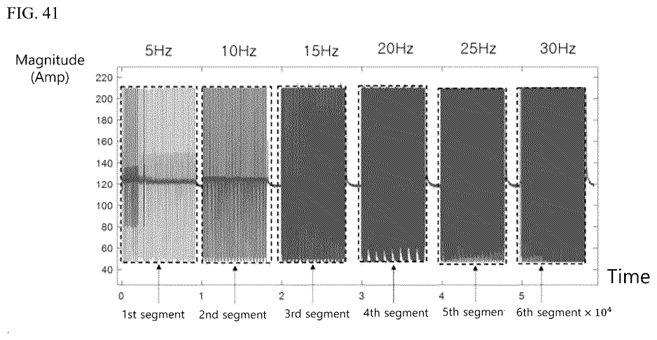

FIG. 41 is a diagram for explaining a method of applying evaluation electrical stimulation (ES) and obtaining a neuromuscular system signal in response thereto according to an embodiment of the present disclosure.

According to another embodiment of the present disclosure, the device (100) may apply electrical stimulation (ES) of different frequencies to the user over time. For example, the device (100) may apply to the user electrical stimulation (ES) of a frequency that increases by a predetermined frequency over time. As a specific example, electrical stimulation (ES) of 5 Hz may be applied during a first time unit, electrical stimulation (ES) of 10 Hz during a second time unit, and electrical stimulation (ES) of 15 Hz during a third time unit.

In this case, the stimulated muscle contraction signal (SMCS) acquired by the device (100) may also comprise different frequencies over time. For example, a stimulated muscle contraction signal (SMCS) received in response to electrical stimulation (ES) of 5 Hz may comprise a frequency of 5 Hz, a stimulated muscle contraction signal (SMCS) received in response to electrical stimulation (ES) of 10 Hz may comprise a frequency of 10 Hz, and a stimulated muscle contraction signal (SMCS) received in response to electrical stimulation (ES) of 15 Hz may comprise a frequency of 15 Hz.

The device (100) may divide the acquired neuromuscular system signal into a plurality of segment units based on frequency. For example, referring to FIG. 12, the device (100) may divide the acquired neuromuscular system signal into a plurality of segments. In this case, signals included in each segment may have the same frequency. Also, in this case, signals included in each segment may be signals having frequencies within a specific range.

According to another embodiment of the present disclosure, the device (100) may acquire a neuromuscular system signal and analyze frequencies comprised in the acquired neuromuscular system signal. Also, the device (100) may divide the neuromuscular system signal into a plurality of segment units by extracting signals for each frequency of a predetermined unit based on the analyzed frequencies.

Those of ordinary skill in the art of the present disclosure will understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of ordinary skill in the art of the present disclosure will understand that the various illustrative logical blocks, modules, processors, means, circuits, and algorithm steps described in connection with the embodiments disclosed herein may be implemented as electronic hardware, various forms of program or design code (referred to herein as software, for convenience), or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Those of ordinary skill in the art of the present disclosure may implement the described functionality in varying ways for each particular application, but such implementation decisions should not be interpreted as causing a departure from the scope of the present disclosure.

The various embodiments presented herein may be implemented as a method, apparatus, or article of manufacture using standard programming and/or engineering techniques. The term article of manufacture includes a computer program, carrier, or media accessible from any computer-readable storage device. For example, computer-readable storage media include, but are not limited to, magnetic storage devices (e.g., hard disks, floppy disks, magnetic strips, etc.), optical disks (e.g., CDs, DVDs, etc.), smart cards, and flash memory devices (e.g., EEPROMs, cards, sticks, key drives, etc.). In addition, various storage media presented herein include one or more devices and/or other machine-readable media for storing information.

It is to be understood that the specific order or hierarchy of steps in the processes presented is an example of illustrative approaches. It is to be understood that, based on design priorities, the specific order or hierarchy of steps in the processes may be rearranged within the scope of the present disclosure. The accompanying method claims provide elements of the various steps in a sample order, but are not meant to be limited to the specific order or hierarchy presented.

The description of the presented embodiments is provided to enable any person of ordinary skill in the art of the present disclosure to use or practice the present disclosure. Various modifications to these embodiments will be readily apparent to those of ordinary skill in the art of the present disclosure, and the general principles defined herein may be applied to other embodiments without departing from the scope of the present disclosure. Thus, the present disclosure is not intended to be limited to the embodiments presented herein, but is to be accorded the widest scope consistent with the principles and novel features presented herein.

What is claimed is:

1. A method of performing neuromuscular system condition evaluation by a device, the method comprising:
obtaining a neuromuscular system signal obtained in response to electrical stimulation of a plurality of frequencies;
obtaining a feature vector by analyzing the neuromuscular system signal in at least one domain among a frequency domain, a time domain, and a time-frequency domain; and
performing neuromuscular system condition evaluation by obtaining a classification result by inputting the feature vector obtained from the neuromuscular system signal into an artificial intelligence model trained by feature vectors representing characteristics of muscles.

2. The method of claim 1, wherein the obtaining of the feature vector comprises:
segmenting the neuromuscular system signal into a plurality of segments based on frequency;
transforming the neuromuscular system signal into a time-frequency domain for each of the segmented plurality of segments;
generating an envelope from the transformed neuromuscular system signal for each of the segmented plurality of segments; and
obtaining a feature vector for each segment by analyzing a pattern of the generated envelope.

3. The method of claim 2, wherein the generating of the envelope comprises:
obtaining envelope peaks by using a positive peak and a negative peak; and
generating the envelope by interpolation between the envelope peaks,
wherein the obtaining of the envelope peaks is performed by:
(1) obtaining the envelope peak by adding magnitude values of an adjacent positive peak and negative peak;
(2) obtaining the envelope peak by calculating a mean of magnitude values of an adjacent positive peak and negative peak;
(3) obtaining the positive peak as the envelope peak; or
(4) obtaining the negative peak as the envelope peak.

4. The method of claim 3, wherein the analysis of the pattern of the envelope is performed by at least one of sample entropy analysis, permutation entropy analysis, standard deviation signal analysis, root mean square analysis, auto-correlation analysis, cardinality analysis, and Poincaré plot analysis.

5. The method of claim 3, wherein the device generates the envelope by applying at least one of linear interpolation, parabolic interpolation, Newton interpolation, Lagrange interpolation, and spline interpolation.

6. The method of claim 1, wherein the obtaining of the feature vector comprises:
performing a Fourier Transform on the neuromuscular system signal to generate FT neuromuscular system signal information;
calculating a log spectrum of the neuromuscular system signal by applying a logarithmic function to the FT neuromuscular system signal information;
obtaining CA (cepstrum analysis) coefficient information by performing an Inverse Fourier Transform on the calculated log spectrum; and
obtaining a feature vector based on the CA coefficient information.

7. The method of claim 6, wherein the obtaining of CA (cepstrum analysis) coefficient information by performing the Inverse Fourier Transform is performed by:
(1) obtaining the CA coefficient information based on a complex value among a complex value and a real value;
(2) obtaining the CA coefficient information based on a real value among a complex value and a real value; or
(3) obtaining the CA coefficient information based on a value obtained through a root operation after squaring and adding a complex value and a real value, respectively.

8. The method of claim 7, wherein the obtaining of the feature vector comprises:
generating preprocessed CA coefficient information by removing an FF (fundamental frequency) component corresponding to a frequency of the electrical stimulation and an HF (harmonic frequency) component generated by the electrical stimulation from the CA coefficient information, and obtaining a feature vector based on the preprocessed CA coefficient information.

9. The method of claim 8, wherein the obtaining of the feature vector comprises:
obtaining temporal change statistics information of the preprocessed CA coefficient information, and obtaining a feature vector based on the temporal change statistics information.

10. The method of claim 9, wherein the temporal change statistics information is determined based on at least one of a maximum value, a minimum value, a mean value, a median value, a standard deviation value, a variance value, and an IQR (inter-quartile range) value of the preprocessed CA coefficient information over time.

11. The method of claim 1, wherein:
the electrical stimulation of the plurality of frequencies is a plurality of sets of electrical stimulation, one set of electrical stimulation is a plurality of electrical stimulations having an identical frequency and an identical intensity, and the plurality of sets of electrical stimulation each have a different frequency, and
the method comprises:
obtaining a neuromuscular system signal for each of the plurality of sets of electrical stimulation generated by applying the plurality of sets of electrical stimulation to a muscle, generating comparison data of at least a portion of a signal of an initial part and at least a portion of a signal of a later part of the neuromuscular system signal, generating a feature vector based on the generated comparison data, and evaluating a condition of the neuromuscular system by inputting the generated feature vector into an artificial intelligence model trained by feature vectors representing characteristics of muscles.

12. The method of claim 11, wherein a stimulation within a predetermined number of times of one set of electrical stimulation is the initial part, and a stimulation after the predetermined number of times of the one set of electrical stimulation is the later part.

13. The method of claim 12, wherein the comparison data is data generated by comparison between a neuromuscular system signal obtained in response to a first stimulation of the one set of electrical stimulation and a neuromuscular system signal obtained in response to a last stimulation of the one set of electrical stimulation.

14. The method of claim 13, wherein the comparison data is generated based on:
(1) an amplitude of a signal;
(2) zero crossing data, which is frequency data regarding the number of times a signal crosses a horizontal line;
(3) mean absolute value data, which is an indicator representing a mean of absolute values of a signal;
(4) slope sign change data, which is data regarding the number of times a sign of a slope of a signal changes;
(5) Willison amplitude data, which is data regarding a degree to which changes equal to or greater than a given threshold value appear in a signal; or
(6) variance data, which is data representing a variability of a data set.

15. The method of claim 1, wherein the obtaining of the feature vector comprises:
generating a first feature vector by preprocessing the obtained neuromuscular system signal through a first preprocessing method; and
generating a second feature vector by preprocessing the obtained neuromuscular system signal through a second preprocessing method,
wherein the performing of the neuromuscular system condition evaluation comprises performing neuromuscular system condition evaluation by obtaining a classification result by inputting the first feature vector and the second feature vector into an artificial intelligence model trained by feature vectors representing characteristics of muscles, and
wherein the first preprocessing method and the second preprocessing method are different.

16. The method of claim 15, wherein the method of performing the neuromuscular system condition evaluation comprises:
evaluating a condition of the neuromuscular system by inputting personal information of a user together with the first feature vector and the second feature vector into an artificial intelligence model, and the personal information of the user comprises at least one of weight information, age information, height information, gender information, medical history information, and medical data of the user.

17. The method of claim 16, wherein:
the first preprocessing method preprocesses the neuromuscular system signal in a time domain or a frequency domain, and
the second preprocessing method preprocesses the neuromuscular system signal by transforming one-dimensional data in a time domain into two-dimensional data in a time-frequency domain.

18. The method of claim 17, wherein the first preprocessing method comprises a cepstrum method, and the cepstrum method comprises:
performing a Fourier Transform on the neuromuscular system signal to generate FT neuromuscular system signal information;
calculating a log spectrum of the neuromuscular system signal by applying a logarithmic function to the FT neuromuscular system signal information;
obtaining CA (cepstrum analysis) coefficient information by performing an Inverse Fourier Transform on the calculated log spectrum; and
obtaining a feature vector based on the CA coefficient information.

19. The method of claim 17, wherein the second preprocessing method obtains the second feature vector using a 2D-autoencoder method or a pattern extraction method, and the pattern extraction method comprises:
segmenting the neuromuscular system signal into a plurality of segments based on frequency;
transforming the neuromuscular system signal into a time-frequency domain for each of the segmented plurality of segments;
generating an envelope from the transformed neuromuscular system signal for each of the segmented plurality of segments; and
obtaining a feature vector for each segment by analyzing a pattern of the generated envelope.

20. A non-transitory computer-readable storage medium storing a computer program comprising instructions for causing one or more processors to perform neuromuscular system condition evaluation, the instructions comprising:
obtaining a neuromuscular system signal obtained in response to electrical stimulation of a plurality of frequencies;
obtaining a feature vector by analyzing the neuromuscular system signal in at least one domain among a frequency domain, a time domain, and a time-frequency domain; and
performing neuromuscular system condition evaluation by obtaining a classification result by inputting the feature vector obtained from the neuromuscular system signal into an artificial intelligence model trained by feature vectors representing characteristics of muscles.
